(19) United States
(12) Patent Application Publication
(10) Pub. No.: US 2025/0188149 A1
(43) Pub. Date: Jun. 12, 2025

(54) ENHANCING ESTROGEN RECEPTOR ALPHA IN OSTEOARTHRITIS

(71) Applicant: University of Pittsburgh - Of the Commonwealth System of Higher Education, Pittsburgh, PA (US)

(72) Inventors: Hang Lin, Wexford, PA (US); Ning Wang, Changsha City (CN)

(21) Appl. No.: 18/843,921

(22) PCT Filed: Mar. 8, 2023
(86) PCT No.: PCT/US2023/014792

§ 371 Date: Sep. 4, 2024

Related U.S. Application Data
(60) Provisional application No. 63/317,724, filed on Mar. 8, 2022.

(51) Int. Cl. C07K 14/72; A61K 31/138; A61K 31/7068; A61K 31/713; C12N 15/113

(52) U.S. Cl. CPC C07K 14/721; A61K 31/138; A61K 31/7068; A61K 31/713; C12N 15/113; C12N 2310/14

(57) ABSTRACT

A method of treating osteoarthritis includes an injector system comprising an agent to increase estrogen receptor-alpha in affected cartilage wherein the agent is delivered locally to the affected cartilage. The agent to increase estrogen receptor-alpha comprises at least one of an agent to effect knock-in of an estrogen receptor-alpha gene, an agent to effect interference with microRNA which suppress estrogen receptor-alpha gene expression, or an agent to effect enhancement of estrogen receptor-alpha gene expression.

### CROSS-REFERENCE TO RELATED APPLICATIONS

This application claims benefit of U.S. Provisional Patent Application Ser. No. 63/317,724, filed Mar. 8, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Osteoarthritis (OA) is a painful and disabling joint disease that impairs patients' life quality. Currently, there is no regulatory-body-approved treatments that can halt or reverse the progression of OA. Loss of articular cartilage is the most salient feature of osteoarthritis (OA), but pathological changes to other joint elements have also been observed, which together result in joint dysfunction with patient pain and disability. While the pathogenesis of OA is multifactorial, mechanical overload of the articular surface, whether sustained acutely in trauma or chronically due to joint malalignment, instability, or obesity, is a known driver of OA onset and progression. Particularly, the term “mechanoflammation” is often used to describe the fact that mechanical stress can drive inflammatory signaling, which directly induces cartilage degradation. Oxidative stress and other signaling pathways have also been shown to mediate the mechanical overload-induced OA progression. Chondrocyte senescence, characterized by permanent cell cycle arrest and the release of pro-inflammatory molecules constituting the senescence-associated secretory phenotype (SASP), has been considered an important feature in OA cartilage.

### SUMMARY

In one aspect, a method of treating osteoarthritis include increasing estrogen receptor-α in affected cartilage. Estrogen receptor-α may, for example, be increased via delivery of at least one of an agent to effect knock-in of an estrogen receptor-α gene, an agent to effect interference with microRNA which suppress estrogen receptor-α gene expression, or an agent to effect enhancement of estrogen receptor-α gene expression. A single agent may, for example, achieve more than one such affect.

Gene therapy may be used in increasing estrogen receptor a. Gene therapy methodologies may, for example, include introduction of specific cell function-altering genetic material. Gene therapy may, for example, include delivery of genetic material to target tissue/cells via a vector such as DNA plasmid or a viral vector. Examples of such viral vectors include, but are not limited to, adeno-associated-virus (AAV) vectors, adenovirus vectors, or lentivirus vectors. An agent to effect knock-in of an estrogen receptor-α gene may, for example, include delivery of gene delivery vector (for example, a plasmid DNA or a viral vector). The vector may include an estrogen receptor-α gene. The agent to effect knock-in of an estrogen receptor-α gene may, for example, further include one or more transduction agents.

In a number of embodiments, an agent to effect interference with microRNA which suppress estrogen receptor-α gene expression includes siRNA. In a number of embodiments, an agent to effect enhancement of estrogen receptor-α gene expression includes a small molecule compound. In general, as used herein small molecule compounds may, for example, have a molecular weight below 1.5 kDa or below 1.0 kDa. In a number of embodiments, the agent to effect enhancement of estrogen receptor-α gene expression is a peptide or a selective estrogen receptor modulator. In a number of embodiments, the selective estrogen receptor modulator may, for example, include or be 4-hydroxytamoxifen or 5-aza-2-deoxycytidine.

In a number of embodiments, the agent to increase estrogen receptor-α in affected cartilage is delivered locally to the affected cartilage. In a number of embodiments, the at least one of the agent to effect at least one of knock-in of an estrogen receptor-α gene, the agent to effect interference with microRNA which suppress estrogen receptor-α gene expression, or the agent to effect enhancement of estrogen receptor-α gene expression is delivered locally to the affected cartilage.

In a further aspect, a system for treating osteoarthritis includes a delivery system such as an injector system and an agent to increase estrogen receptor-α in affected cartilage within a reservoir of the injector system. As described above, estrogen receptor-α may, for example, be increased via delivery of at least one of an agent to effect knock-in of an estrogen receptor-α gene, an agent to effect interference with microRNA which suppress estrogen receptor-α gene expression, or an agent to effect enhancement of estrogen receptor-α gene expression. A single agent may, for example, achieve more than one such affects.

The agent to effect knock-in of an estrogen receptor-α gene may, for example, include delivery of a vector for gene delivery to the target tissue/cells. Such a vector may, for example, be a plasmid DNA or a viral vector. The vector may include an estrogen receptor-α gene. The agent to effect knock-in of an estrogen receptor-α gene may, for example, further include one or more transduction agents.

In a number of embodiments, the agent to effect interference with microRNA which suppress estrogen receptor-α gene expression includes siRNA. In a number of embodiments, the agent to effect enhancement of estrogen receptor-α gene expression includes a small molecule compound. In general, as used herein small molecule compounds may, for example, have a molecular weight below 1.5 kDa or below 1.0 kDa. In a number of embodiments, the agent to effect enhancement of estrogen receptor-α gene expression is a peptide or a selective estrogen receptor modulator. In a number of embodiments, the selective estrogen receptor modulator may, for example, include or be 4-hydroxytamoxifen or 5-aza-2-deoxycytidine.

In a number of embodiments, the delivery system (for example, an injection system) is configured to deliver the agent to increase estrogen receptor-α in affected cartilage locally to the affected cartilage. The injection system may, for example, be configured to deliver the at least one of the agent to effect at least one of knock-in of an estrogen receptor-α gene, the agent to effect interference with microRNA which suppress estrogen receptor-α gene expression, or the agent to effect enhancement of estrogen receptor-α gene expression locally to the affected cartilage.

The present devices, systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a schematic showing the process of collecting osteochondral samples. Discarded femoral and tibial surfaces from total knee arthroplasty (TKA) were used to harvest P-C and D-C together with the subchondral bone. P-C and D-C were delineated by Outerbridge scoring, based on macroappearance, and OARSI scoring, based on histological staining. The chondrocytes were isolated from P-C (P-CHs) or D-C (D-CHs) and expanded in vitro. Afterward, chondrocytes were subjected to pellet cultured in chondrogenic medium.

FIG. 1B illustrates P-C and D-C samples which were harvested from TKA surgical waste (i-v). In each representative panel, the left and right panels show the articular surface before and after plug harvesting, respectively.

FIG. 1C illustrates Safranin O/Fast green staining for representative (i-iii) P-C and (iv-vi) D-C. Bar=500 μm.

FIG. 1D illustrates information of six donors. Outerbridge and OARSI scores were used to define the P-C and D-C samples. Average Outerbridge scores for P-C was 0.6 and for D-C was 2.8; OARSI histopathology scores for P-C and D-C from six donors. Average OARSI score for P-C was 6.8, and for D-C was 19.7.

FIG. 2A illustrates safranin O/Fast green staining of P-C and D-C samples.

FIG. 2B illustrates MMP13 of P-C and D-C samples.

FIG. 2C illustrates p16^{INK4a} (a protein that slows cell division by slowing the progression of the cell cycle from the G1 phase to the S phase) immunohistochemistry (IHC) of P-C and D-C samples.

FIG. 2D illustrates β-Galactosidase staining for P-C (i, iii, v, vii) and D-C (ii, iv, vi, viii). Bar=50 μm. Arrows indicate positive staining. Images with lower magnification are also included on the left bottom corner.

FIG. 2E illustrates qRT-PCR analysis of expression levels of the representative genes associated with chondrogenesis, senescence, inflammation, fibrogenesis, osteogenesis, degradation and hypertrophy in P-CHs and D-CHs at passage 0 (P0). All data were normalized to those from the P-CH group. (COL2: Collagen type 2, SOX9: SRY-Box Transcription Factor 9, ACAN: Aggrecan, p21: cyclin-dependent kinase inhibitor 1, p53: tumor suppressor p53, IL6: Interleukin 6, IL8: Interleukin 8, COL1: Collagen type 1, COL3: Collagen type 3, VCAN: Versican, OCN: Osteocalcin, OPN: Osteopontin, OSX: Osterix, RUNX2: Runt-related transcription factor 2, VEGF: Vascular endothelial growth factor, ADAMTS4: ADAM Metallopeptidase With Thrombospondin Type 1 Motif 4, ADAMTS5: ADAM Metallopeptidase With Thrombospondin Type 1 Motif 5. MMP12: Matrix Metalloproteinase 12, COL10: Collagen type 10, ALP: Alkaline phosphatase). N=18 per group, **, p<0.01, ***, p<0.001, ****, p<0.0001.

FIG. 2F illustrates protein expression levels of p16^{INK4a}, p21, p53, MMP13, in P0 P-CHs and D-CHs, which were studied with western blot.

FIG. 3A illustrates safranin O/Fast green staining and western blot for cartilaginous pellet derived from P-CHs and D-CHs, Bar=50 μm.

FIG. 3B illustrates schematically flow for silencing p16^{INK4a} in D-CHs and evaluating the influences on two-dimensional (2D) and 3D culture.

FIG. 3C illustrates SA-β-Gal staining for D-CHs on 2D culture, bar=50 μm.

FIG. 3D illustrates results of qRT-PCR to analyze the expression levels of marker genes that are associated with senescence, chondrogenesis, inflammation and degradation. All data was normalized to those from the siCON group. N=4 per group; *, p<0.05; **, p<0.01.

FIG. 3E illustrates safranin O/Fast green staining of pellet generated from siCON D-CHs and sip16^{INK4a} D-CHs.

FIG. 3F illustrates protein expression levels of senescence-relative markers and cartilage degradation-relative markers in 2D and 3D culture.

FIG. 4A illustrates the results of RNA-Seq to examine the role of transcriptional factor ERα in OA progression, setting forth the top 5 upstream regulators of DEGs, predicted by IPA (Qiagen), activation Z-score, overlapping p-value, and targeted molecules of each regulator.

FIG. 4B ESR1 gene expression levels in two other published studies Raw RNA-seq data (E-MTAB-4304) were downloaded from GEO database and analyzed with the same pipeline as our RNA-seq data. Ramos Y F, den Hollander W, Bovee J V, Bomer N, van der Breggen R, Lakenberg N, et al. Genes involved in the osteoarthritis process identified through genome wide expression analysis in articular cartilage; the RAAK study. PLoS One 2014; 9: e103056; Dunn S L, Soul J, Anand S, Schwartz J M, Boot-Handford R P, Hardingham T E. Gene expression changes in damaged osteoarthritic cartilage identify a signature of non-chondrogenic and mechanical responses. Osteoarthritis Cartilage 2016; 24: 1431-1440.

FIG. 4C ESR1 gene expression levels in two other published studies Raw RNA-seq data (E-MTAB-4304) were downloaded from GEO database and analyzed with the same pipeline as our RNA-seq data.

FIG. 4D illustrates the genes that are predicted to be regulated by ESR1 in IPA. Genes relevant to cartilage degradation are encircled in thickened line.

FIG. 4E illustrates the protein levels of ERα in representative human P-C and D-C samples were examined with immunohistochemistry. Arrows indicate positive staining. Bar50 μm.

FIG. 4F illustrates the glycosaminoglycans (GAG) content and ERα level in preserved and damaged cartilage isolated from age-matching old mice. Bar=50 μm. Arrows indicate the positive staining from immunohistochemistry for ERα.

FIG. 4G illustrates results qRT-PCR to detect expression levels of ESR1 in P-CHs and D-CHs isolated from 6 donors. Data were normalized to gene expression levels in P-CHs. N=4 per group; *, P<0.05; **, P<0.01; **** P<0.0001.

FIG. 4H ERα protein level in P0 P-CHs and D-CHs was assessed with western blot.

FIG. 4I illustrates the results of Western blot studies wherein P-CHs were transfected with control siRNA (siCON) or ESR1 siRNA (siESR1). siESR1 P-CHs were then further infected with vectors carrying ESR1 gene (kiESR1) in any effort to reverse the knockdown effect of siRNA. Western blot was used to examine the expression levels of representative chondrogenesis, senescence, and degradation-relevant markers in these cells.

FIG. 4J illustrates the results of Western blot studies wherein D-CHs were infected with vectors carrying GFP (Control) or ESR1 (kiESR1) gene. Western blot was used to examine the levels of selected proteins.

FIG. 5A illustrates schematically the application of mechanical loading. P-CHs were encapsulated into gelatin scaffolds that were cylindrically shaped with 2 mm thickness and 3.5 mm diameter. Through controlling the travel distance of the loading piston, 5% and 20% compressive strain were applied, representing physiological or supraphysiological (i.e., injurious) mechanical loading, respectively. The samples in static culture were used as the control (0% group). P-CHs transduced with control (siCON group) or ESR1 (siESR1 group) siRNA were used.

FIG. 5B illustrates the results of qRT-PCR in examining gene expression levels. All data were normalized to the 0% siCON group. N=4 per group; *, P<0.05; ***, P<0.001; ****, P<0.0001.

FIG. 5C illustrates the results of immunofluorescence (IF) in examining ERα levels. The intensity of staining was semi-quantified using Image J. N=4 per group; ****p<0.0001. Bar=50 μm. Green=ERα, Blue=nucleus.

FIG. 5D illustrates studies of immunofluorescence and (F) Bar=50 μm; p16^{INK4a} (upper) or MMP13 (lower).

FIG. 5E illustrates intensity of staining in FIG. 5D which was semi-quantified using Image J. N=4 per group; *, p<0.05; **, p<0.01; ***, p<0.001; ****, p<0.0001.

FIG. 5F illustrates expression levels of GAPDH, p16^{INK4a}, p21, p53, and MMP13 examined with western blot analysis.

FIG. 6A illustrates the results of qRT-PCR to examine representative genes in six groups. N=4 per group; Data were normalized to 0%/siCON group. P-CH were transduced with control (siCON), or ESR1 siRNA (siESR1), loaded in GelMA scaffold, and then subjected to mechanical loading with 0%, 5% or 20% of strain. After five days, the samples were harvested for analyses as set forth in FIGS. 6A through 6G (in which *p<0.05, **p<0.01, ***p<0.001, ****p<0.0001).

FIG. 6B illustrates the results of immunofluorescence studies used to analyze the expression levels of selective proteins. Red staining indicated the presence of COL10 (upper) or osteocalcin (OCN) (lower); Blue=nucleus.

FIG. 6C illustrates the results of studies in which intensity of staining in immunofluorescence (FIG. 6B) is semi-quantified using Image J.

FIG. 6D illustrates the results of Western blot studies used to analyze the expression levels of selective proteins.

FIG. 6E illustrates expression levels of OCN in human P-C and D-C (upper), wild type (WT) and ESR1 knockout (KOesr1) mice (lower), were examined with immunohistochemistry or immunofluorescence. Dark brown or green staining indicated the presence of OCN. Blue=nucleus.

FIG. 6F illustrates studies based on imaging in of FIG. 6E, wherein the protein levels were semi-quantified through Image J and wherein N=6 per group.

FIG. 6G illustrates studies based on imaging in of FIG. 6E, wherein the protein levels were semi-quantified through Image J and wherein N=4 per group.

FIG. 7A illustrates studies of knee joints which were harvested from mice undergoing Sham or DMM surgery wherein immunofluorescence (IF, Green) was used to assess the levels of p16^{INK4a} and cell nuclei were stained with DAPI (Blue), wherein the mean fluorescence intensity from p16^{INK4a} was quantitated with ImageJ. (n=4 random imaging areas from 4 mice and *, p<0.05.

FIG. 7B illustrates studies of knee joints which were harvested from mice undergoing Sham or DMM surgery wherein immunofluorescence (IF, Green) was used to assess the levels of (C) γH2AX and cell nuclei were stained with DAPI (Blue), wherein the mean fluorescence intensity from γH2AX IF was quantitated with ImageJ. (n=4 random imaging areas from 4 mice) and ***, p<0.001.

FIG. 8A illustrates protein levels of Actin, γH2AX, ERα, p16^{INK4a}, and p21 were examined by western blot in characterization of DOX-induced changes in human chondrocytes wherein doxorubicin (DOX, 100 nM) was used to induce DNA damage in healthy human chondrocytes (DOX group), the treatment lasted for three days, and the cells treated with the vehicle were used as the control (Con group).

FIG. 8B illustrates semi-quantification of protein levels for γH2AX, ERα, p16^{INK4a}, and p21 based on the band intensities from the western blot shown in FIG. 8A, wherein data are normalized to the Con group. (n=3 technical replicates). *, p<0.05; ****p<0.0001.

FIG. 8C illustrated quantification of images of the tail moment from comet assay. (n=12 imaging areas from 3 technical replicates). ***, p<0.001

FIG. 8D illustrates (percentage of SA-β gal positive cells per examining area (560 μm×350 μm) determined from SA-β-Gal staining. (n=3 imaging areas). **, p<0.01; ***, p<0.001;

FIG. 8E illustrates quantification of mean fluorescence intensity based on p16^{INK4a} from IF staining for p16^{INK4a} wherein ell nuclei were stained with DAPI (Blue). (n=4 imaging areas). *, p<0.05.

FIG. 8F illustrates quantification of mean fluorescence intensity based on ERα from IF staining for Erα, wherein ell nuclei were stained with DAPI (Blue). (n=4 imaging areas). ****p<0.0001.

FIG. 9A illustrates protein levels of Actin, ERα, p16^{INK4a}, and p21 examined by western blot in studies of the effect of overexpressing ESR1 in DOX-treated human chondrocytes, wherein all cells were pre-treated with DOX (100 nM) for three days and then transfected with vectors carrying mCherry (CON-KI group) or ESR1 (ESR1-KI group) gene.

FIG. 9B illustrates semi-quantification of protein levels based on the band intensities from the western blot shown in FIG. 9A, wherein data are normalized to the CON-KI group. (n=3 technical replicates. *, p<0.05; **, p<0.01.

FIG. 9C illustrates quantification of mean fluorescence intensity from IF images. (n=3 imaging areas) from IF staining for p16^{INK4a} wherein cell nuclei were stained with DAPI (Blue). *, p<0.05.

FIG. 9D illustrates percentage of SA-β gal positive cells per examining area (560 μm×350 μm) from SA-β-Gal staining images. (n=3 imaging areas). ***, p<0.001

FIG. 9E illustrates quantification of the tail moment from comet assay images showing tail movement. (n=18 imaging areas from 3 technical replicates). *****p<0.0.0001.

FIG. 10A illustrates studies of the effect of overexpressing ESR1 in human chondrocytes harvested from OA cartilage three days after transfection with vectors carrying mCherry (CON-KI group) or ESR1 (ESR1-KI group) gene, wherein the relative gene expression levels of ESR1 in human OA chondrocytes were determined by qPCR. Data are normalized to the CON-KI group. (n=3 technical replicates). *, p<0.05.

FIG. 10B illustrates protein levels of Actin, ERα, p16^{INK4a}, and p21 were examined by western blot.

FIG. 10C illustrates semi-quantification of protein levels based on the band intensities from the western blot shown in FIG. 10B. Data are normalized to the CON-KI group. (n=3 technical replicates). ***, p<0.001; ****, p<0.0001.

FIG. 10D illustrates quantification of mean fluorescence intensity from ERα IF images of IF staining for ERα wherein cell nuclei were stained with DAPI (Blue). (n=4 imaging areas). ***, p<0.001.

FIG. 10E illustrates quantification of mean fluorescence intensity from p16^{INK4a} IF images wherein cell nuclei were stained with DAPI (Blue). (n=4 imaging areas). **, p<0.01.

FIG. 10F illustrates percentage of SA-β gal positive cells per examining area (560 μm×350 μm) from SA-β-Gal staining images for chondrocytes. (n=3 imaging areas). ****, p<0.0001.

FIG. 11A illustrates relative gene expression levels of IL6 and IL8 determined by qPCR for normal human chondrocytes which were treated with DOX (100 nM, DOX group) or vehicle control (Con group) for three days, wherein data are normalized to the respective Con (A) group. (n=3 technical replicates). *, p<0.05; **, p<0.01.

FIG. 11B illustrates relative gene expression level of NF-κB determined by qPCR for normal human chondrocytes which were treated with DOX (100 nM, DOX group) or vehicle control (Con group) for three days, wherein data are normalized to the respective Con (B) group. (n=3 technical replicates). *, p<0.05.

FIG. 11C illustrates protein level of Actin, phosphorylated p65 (p-p65), and total p65 as determined by western blot for normal human chondrocytes treated with DOX (100 nM, DOX group) or vehicle control (Con group) for three days.

FIG. 11D illustrates semi-quantification of p65 and p-p65 levels based on the band intensities from the western blot shown in FIG. 11C, wherein data are normalized to the Con (D) group. (n=3 technical replicates). (n=3 technical replicates). *, p<0.05.

FIG. 11E illustrates relative gene expression levels of IL6 and IL8 examined by qPCR, wherein data are normalized to the respective CON-KI (E) group, wherein normal human chondrocytes treated with DOX (100 nM, DOX group) or vehicle control (Con group) for three days, and wherein normal human chondrocytes were pre-treated with DOX for three days and then transfected with vectors carrying mCherry (CON-KI group) or ESR1 (ESR1-KI group) gene. (n:=3 technical replicates). **, p<0.01; ***, p<0.001.

FIG. 11F illustrates relative gene expression level of NF-κB wherein data are normalized to the respective CON-KI (F) group for normal human chondrocytes treated as described in connection with FIG. 11E. (n=3 technical replicates). **, p<0.01.

FIG. 11G illustrates protein level of Actin, phosphorylated p65 (p-p65), and total p65 determined by western blot for normal human chondrocytes treated as described in connection with FIG. 11E.

FIG. 11H illustrates semi-quantification of p65 and p-p65 levels based on the band intensities from the western blot shown FIG. 11G, wherein data are normalized to the CON-KI (H) group for normal human chondrocytes treated as described in connection with FIG. 11E. (n=3 technical replicates). *, p<0.05.

FIG. 11I illustrates proposed interactions of ERα with DNA damage and cellular senescence in chondrocytes.

FIG. 12 illustrates Western blot studies demonstrating that 4-hydroxytamoxifen can increases ERα levels.

FIG. 13 illustrates an embodiment of a delivery system for an agent to increase estrogen receptor-α in cartilage affected by osteoarthritis.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a compound” includes a plurality of such compounds and equivalents thereof known to those skilled in the art, and so forth, and reference to “the compound” is a reference to one or more such compound and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

Results of studies of encapsulated human chondrocytes into hyaluronic acid hydrogel and cultured the constructs under dynamic compressive loading indicate that loading with strain larger than 20% induced the expression of senescence-relevant markers. However, the mechanisms by which mechanical loading influences chondrocyte senescence have not been elucidated. Given the association of increased cartilage loss in regions of higher contact stresses in osteoarthritic joints, the analysis of chondrocytes found in severely damaged (that is, degenerated) cartilage as compared to those in preserved cartilage of the same joint permits the investigation of the effects of different mechanical loads in a shared biochemical milieu. In a number of studies hereof, RNA sequencing was used for transcriptome-wide analysis of differential gene expression in chondrocytes isolated from regions of damaged cartilage and preserved cartilage procured from human knees that underwent total knee arthroplasty. Of the differentially expressed genes, it was notable that estrogen receptor-1 (ESR1), a gene encoding estrogen receptor-α (ERα), was significantly downregulated in severely damaged cartilage.

The level of ERα on chondrocytes is decreased in patients with OA, when compared to healthy controls. In addition, in an in vitro model of OA, stimulation of chondrocytes with proinflammatory cytokine interleukin 1 beta (IL-1β) upregulates microRNA 203 (miR-203) expression, which sequentially antagonizes ERα function, resulting in increased inflammation and decreased cell viability and expression of chondrogenic matrix proteins. The studies hereof demonstrate a role for ERα in mediating chondrocyte phenotype. The mechanisms underlying these effects have not previously been well elucidated. Moreover, in this study, ERα was differentially expressed in areas bearing varying loads, so ERα may potentially regulate chondrocyte response to mechanical loading, which has not been previously reported.

Mechanical loading, in the form of hydrostatic pressure, modulates osteogenesis through ERα. Namely, mechanical loading at a defined intensity was found to upregulate in vitro osteogenesis in both osteoblasts and mesenchymal stem cells (MSCs) through cytoskeletal remodeling and non-canonical β-catenin signaling. Such in vitro studies, coupled with an in vivo study using a transgenic ERα−/− knockout mouse, demonstrated that ERα is required for the osteogenic response to mechanical loading in a ligand-independent that is, estrogen-independent) manner. The studies hereof demonstrate mechanotransductive signaling through ERα on a chondrogenic phenotype.

In the studies hereof, it was first demonstrated that ERα is an important regulator of chondrocyte phenotype in OA, including modulating senescence and the production of degradative enzymes. Furthermore, through dynamic compressive loading of three-dimensional (3D) chondrocyte-embedded hydrogels under physiological or injurious strains, it was shown that ERα mediates the mechanotransductive effects on chondrocyte phenotype. Particularly, ERα-depleted chondrocytes respond to injurious mechanical loading by significantly enhancing the expression levels of molecules associated with hypertrophy and osteogenesis, key features found in OA cartilage. Therefore, restoring and maintaining ERα function in chondrocytes presents a therapeutic intervention for OA.

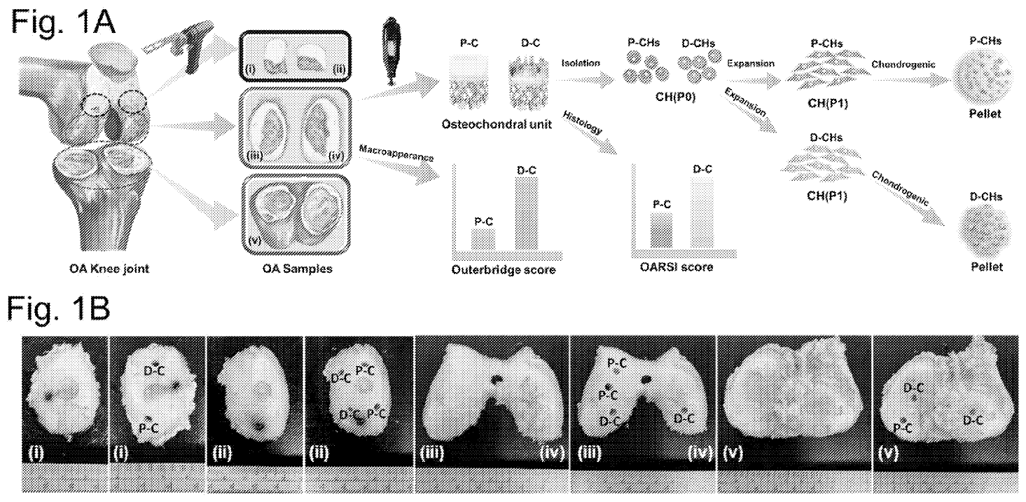

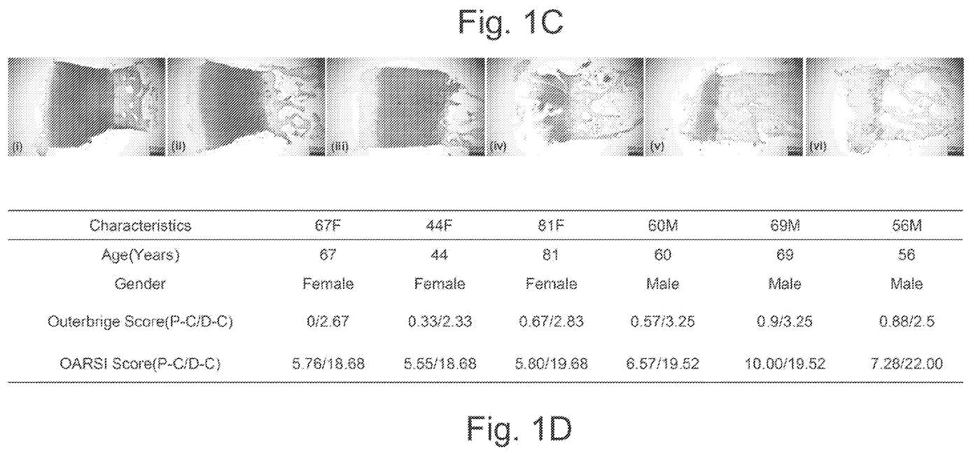

To eliminate the variance due to donor-to-donor disparity, both preserved (P-C) and damaged (D-C) cartilage were harvested from the same knee joint of an OA patient (FIG. 1A). Outerbridge scores assigned by experienced surgeons were used to distinguish P-C and D-C, which were further confirmed by OARSI histopathology grading. Chondrocytes were then isolated from P-C and D-C, and termed P-CHs and D-CHs, respectively. To assess their cartilage-forming capacity, the cells were passaged twice and then cultured as pellets for two weeks in chondrogenic medium. To obtain a sufficient quantity of tissues for multiple analyses and cell culture, tissues were selected from several locations in the knee joint, including both compartments of the femoral condyles and tibial plateau (FIG. 1B). Samples from six donors were used in the studies hereof (FIG. 1D).

Mean Outerbridge scores for P-C and D-C from all six donors are summarized in FIG. 1D. All P-C tissue had minimal to mild macroscopic damage, with a mean Outerbridge score ≤1. In contrast, D-C tissues displayed severe degradation, with a mean Outerbridge score >2. Safranin O/Fast green staining indicated that representative P-C samples (FIG. 1C (i-iii)) displayed strong staining with an intact surface; features not observed in D-C samples (FIG. 1C (iv-v)). The cartilage portion in D-C samples was often thin, with penetrating fissures and uneven surface topology, and weak Glycosaminoglycans (GAGs) staining, representing typical OA features. OARSI histopathology scoring showed that P-C from all samples had a score less than 10, with an average score of 6.8. In contrast, D-C samples had an OARSI score greater than 18, with an average score of 19.7 (FIG. 1D). Intraclass correlation coefficient (ICC) analysis showed good inter-rater reliability for both Outerbridge and OARSI scoring.

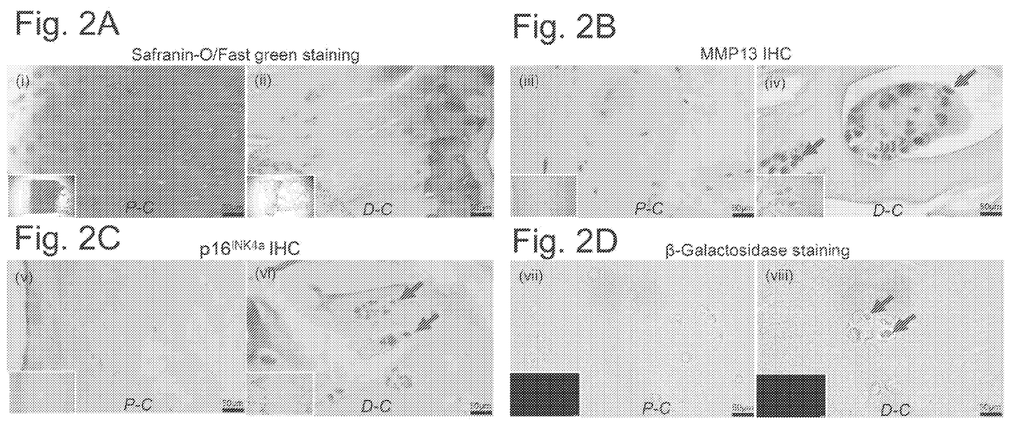

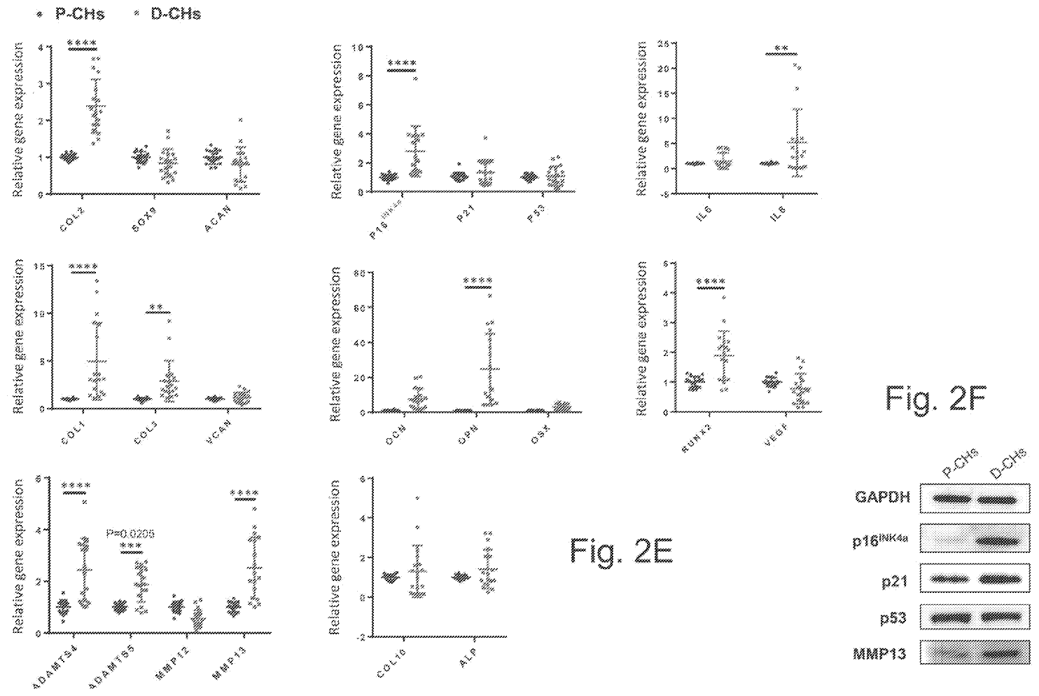

Subsequently, the degradation and senescence-associated markers in the P-C and D-C was examined. Compared to P-C, D-C contained few GAGs (FIG. 2A) and displayed higher levels of Matrix Metallopeptidase 13 (MMP13)(FIG. 2B), p16^{INK4a} (FIG. 2C) and Senescence-associated beta-galactosidase (SA-β-Gal) (FIG. 2D), which indicated that cells in D-C displayed both senescent and OA phenotypes. To confirm the results from histology, chondrocytes (CHs) were isolated from P-C (P-CHs) and D-C (D-CHs), and their phenotypes were examined with real-time quantitative reverse transcription PCR (qRT-PCR) and western blot. To minimize the effect of in vitro expansion on chondrocyte phenotype, cells at passage 0 (P0) were used. In general, D-CHs displayed higher expression levels of senescence, inflammation, fibrogenesis, osteogenesis, and degradation-related genes than P-CHs (FIG. 2E). No significant differences in the expression of representative chondrogenic genes and hypertrophic markers, including Aggrecan (ACAN), collagen type X (COL10), and Alkaline phosphatase (ALP), was observed between P-CHs and D-CHs. In fact, expression level of collagen type II (COL2) was found to be higher in D-CHs than P-CHs.

The senescent and osteoarthritic features in D-CHs were further confirmed by western blot analysis. As shown in FIG. 2F, D-CHs contained higher protein levels of p15^{INK4a}, p21, and MMP13 than P-CHs.

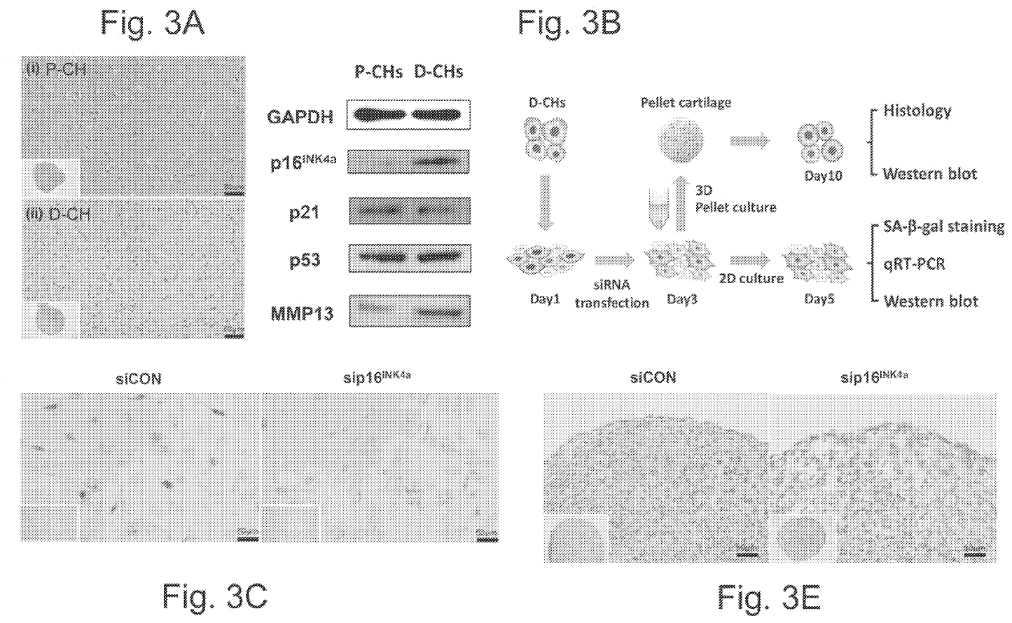

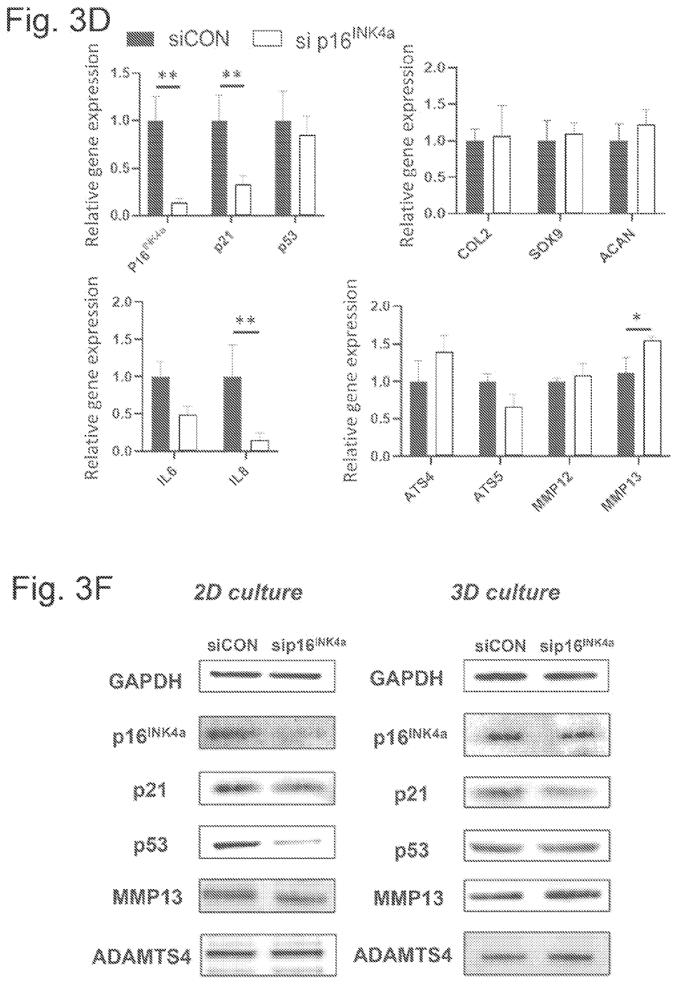

Since chondrocytes play a critical role in maintaining cartilage homeostasis by replenishing the loss of matrix, the cartilage-forming capacity of P-CHs and D-CHs via conventional pellet culture in chondrogenic medium was examined. As shown, for example, in FIG. 3A, the cartilage pellets generated from D-CHs generally inherited the phenotype of D-C, such as low GAG deposition and high level of cellular senescence.

Taken together, compared to P-C and P-CHs-derived cartilage, D-C and D-CHs-derived cartilage displayed enhanced levels of senescence, inflammation, fibrogenesis, osteogenesis, and degradation.

Since p16^{INK4a} was significantly upregulated in D-CHs, we first tested if suppressing p16^{INK4a} could reverse osteoarthritic and senescent phenotypes in D-CHs. Small interfering RNA (sip16^{INK4a}) that targets cyclin-dependent kinase inhibitor 2A (CDKN2A, the gene encoding p16^{INK4a}) was used to reduce p16^{INK4a} level (FIG. 3B). As expected, sip16^{INK4a} treatment markedly decreased the protein levels of p16^{INK4a} (FIG. 3F) on 2D chondrocyte culture, and partially reversed the senescent level (FIGS. 3C and 3D). However, p16^{INK4a}-suppressed D-CHs failed to generate cartilage upon chondrogenic stimulation, indicated by low expression of chondrogenic genes (FIG. 3D) and weak GAG staining (FIG. 3E). Moreover, suppressing p16^{INK4a} promoted MMP13 expression in pellet culture (FIGS. 3D and 3F). These results indicated that p16^{INK4a} might not be the primary factor driving the osteoarthritic conversion of chondrocytes.

To identify the factor (s) that results in the difference between P-C and D-C, RNA-Seq analysis was performed to examine the transcriptome of P-CHs and D-CHs. Gene counts across all samples were quantified and normalized. For pairwise comparison, 313 differentially expressed genes (DEGs) that were up-regulated were identified and 245 DEGs that were down-regulated were identified. Based on identified DEGs, Gene Ontology (GO) enrichment analysis, Kyoto Encyclopedia of Genes and Genomes (KEGG) and Reactome pathway enrichment analysis were conducted. KEGG is a web-based collection of databases for genomes, biological pathways, drugs and chemical substances. The Reactome pathway database is a web-based, open source, curated and peer-reviewed pathway database. Results revealed that the cartilage degradation-relevant genes were most changed in D-CHs.

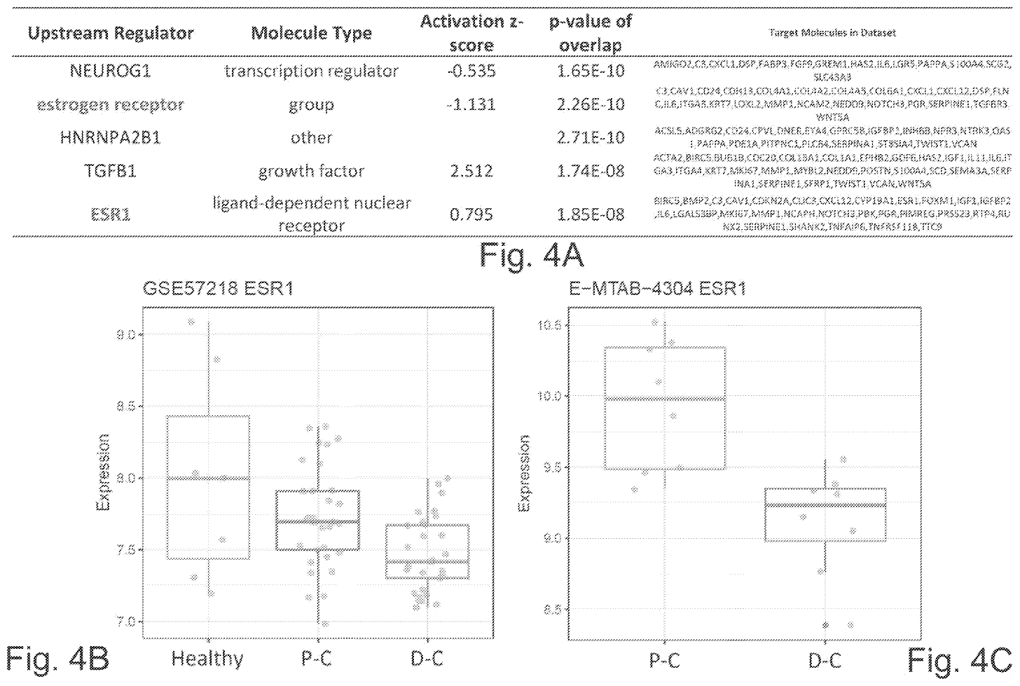

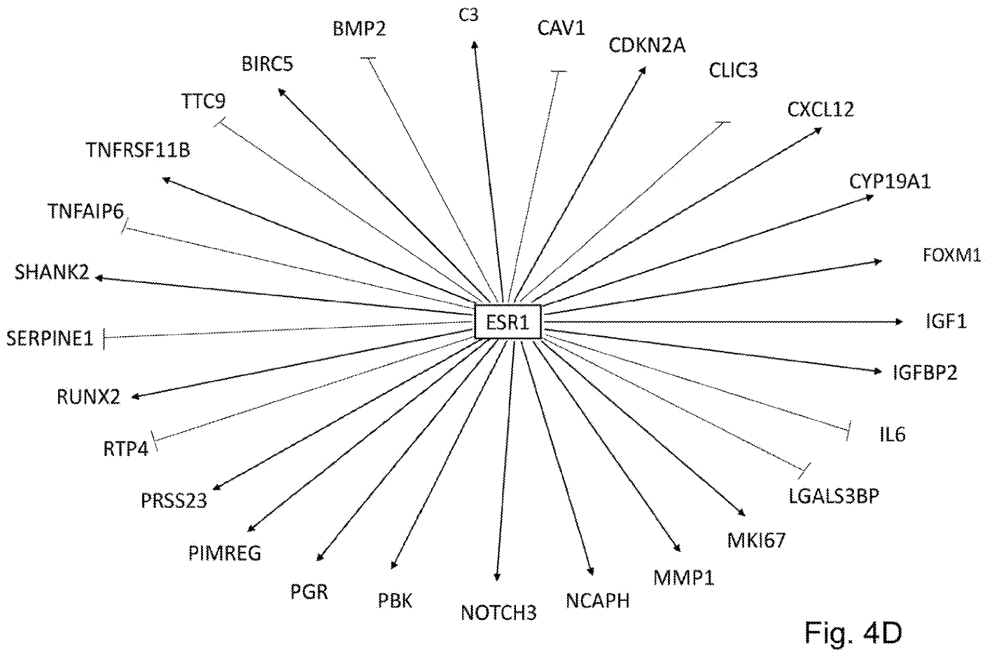

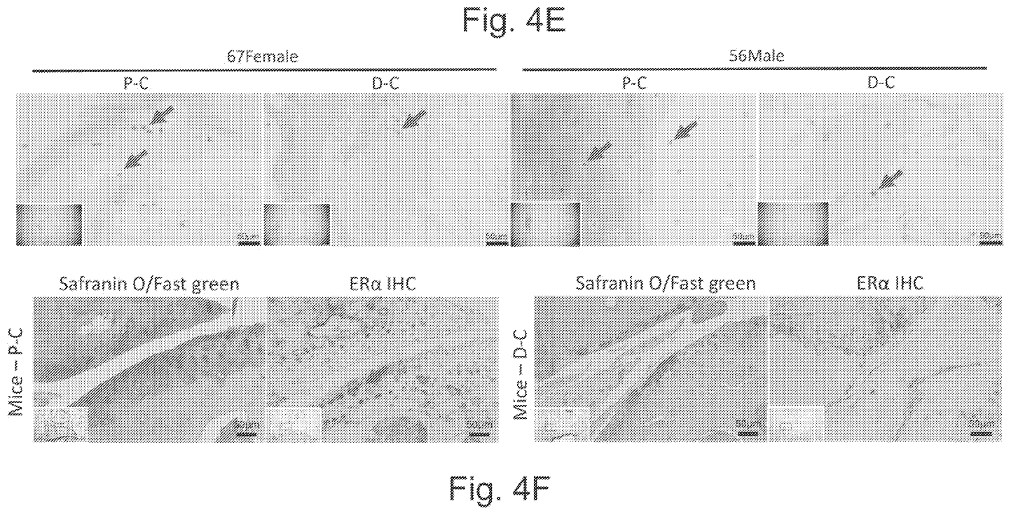

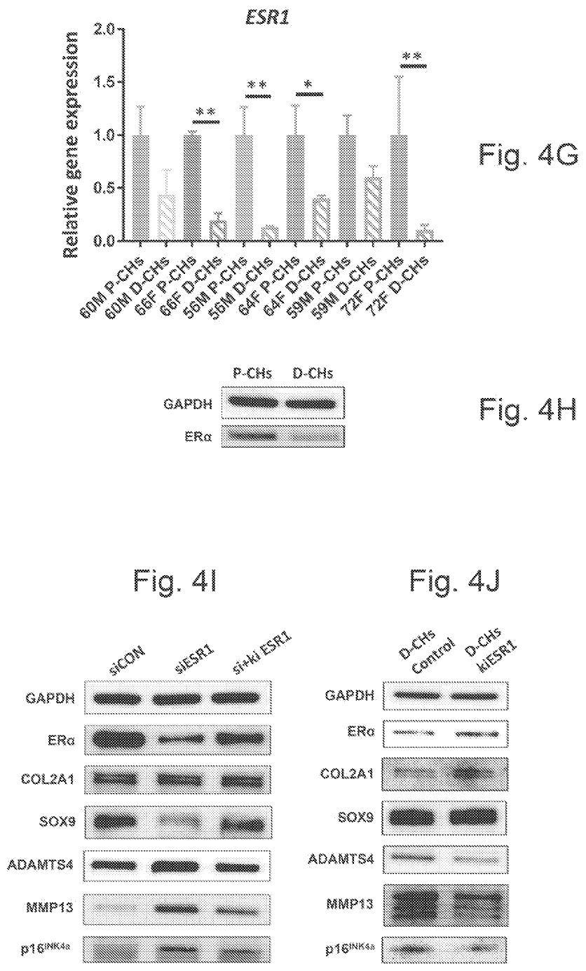

Based on the DEGs described above, Ingenuity pathway analysis (IPA) was performed. IPA is a web-based software application providing for analysis, integration, and understanding of data from gene expression, miRNA, and SNP microarrays, as well as metabolomics, proteomics, and RNAseq experiments (available from QIAGEN, Venlo, Netherlands). The top 5 upstream regulators predicted by IPA are listed in FIG. 4A, with their activation Z-score, overlapping p-value and their target molecules. Estrogen receptor family and estrogen receptor-1 (ESR1), a gene encoding estrogen receptor-α (ERα), both ranked in the top 5 upstream regulators, which strongly suggested an important role for regulating DEGs. Compared to P-CHs, D-CHs displayed significantly reduced expression of ESR1. Similar results were also derived from additional analysis by the present inventors of open data from two published studies (GSE57218, E-MTAB-4304) See Ramos Y F, den Hollander W, Bovee J V, Bomer N, van der Breggen R, Lakenberg N, et al. Genes involved in the osteoarthritis process identified through genome wide expression analysis in articular cartilage; the RAAK study. PLoS One 2014; 9: e103056; Dunn S L, Soul J, Anand S, Schwartz J M, Boot-Handford R P, Hardingham T E. Gene expression changes in damaged osteoarthritic cartilage identify a signature of non-chondrogenic and mechanical responses. Osteoarthritis Cartilage 2016; 24: 1431-1440; and den Hollander W, Ramos Y F, Bos S D, Bomer N, van der Breggen R, Lakenberg N, et al. Knee and hip articular cartilage have distinct epigenomic landscapes: implications for future cartilage regeneration approaches. Ann Rheum Dis 2014; 73: 2208-2212. (FIGS. 4B and 4C). The downstream targets of ERα were also examined. As shown in FIG. 4D, many genes related to cartilage function and OA were predicted to be regulated by ERα, including p16^{INK4a} (CDKN2A).

To confirm the findings from the RNA-Seq, the level of ERα were examined in different samples. Results from immunohistochemistry indicated lower expression of ERα in D-C from all six donors when compared to respectively corresponding P-C (FIG. 4E). Such a difference was observed in both genders. Similar reduction of ERα was also observed in preserved and damaged cartilage isolated from mice, indicating a conserved mechanism across species (FIG. 4F). The gene expression level of ESR1 and ERα protein level in isolated P-CHs and D-CHs from all donors were also examined (FIGS. 4G and 4H). Results confirmed the significant decrease of ERα level in D-CHs.

To understand the role of ERα, gain- and loss-of-function experiments were performed in P-CHs and D-CHs. Knockdown of ESR1 in P-CHs through siRNA resulted in elevated levels of p16^{INK4A}, MMP13, and ADAMTS4, which were concomitant with decreased levels of SOX9 (FIG. 4I). The effects of ERα suppression on P-CHs were able to be reversed by transducing ESR1 gene with lentiviral vectors (FIG. 4I).

Since D-CHs displayed low ERα expression, it was subsequently tested if elevating ERα could reverse the senescent and OA phenotype of D-CHs. Enhancing ERα level via transducing ESR1 gene not only reduced the senescence level and increased proliferation potential, respectively indicated by reduced expression of p16^{INK4A} and increased cell numbers and ki67 levels (FIG. 4J), but also ameliorated OA phenotype, revealed by increased expression of COL2, and reduced expression of MMP13 and ADAMTS4. Serum- and phenol red-free medium were used in these studies, and no estrogen or other potential ERα activators were added as medium supplements. Therefore, the results indicate that ERα regulates chondrocyte phenotype without the need for estrogen.

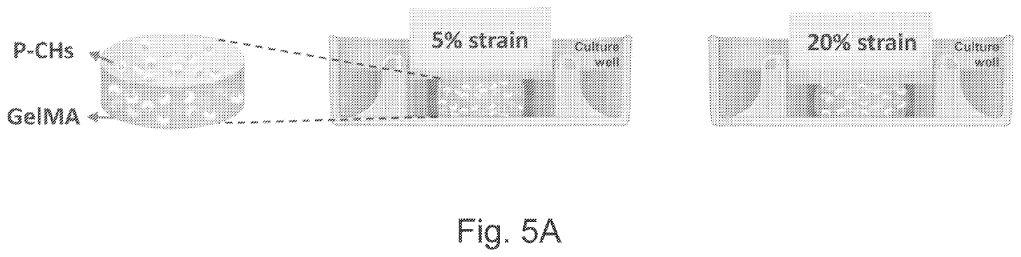

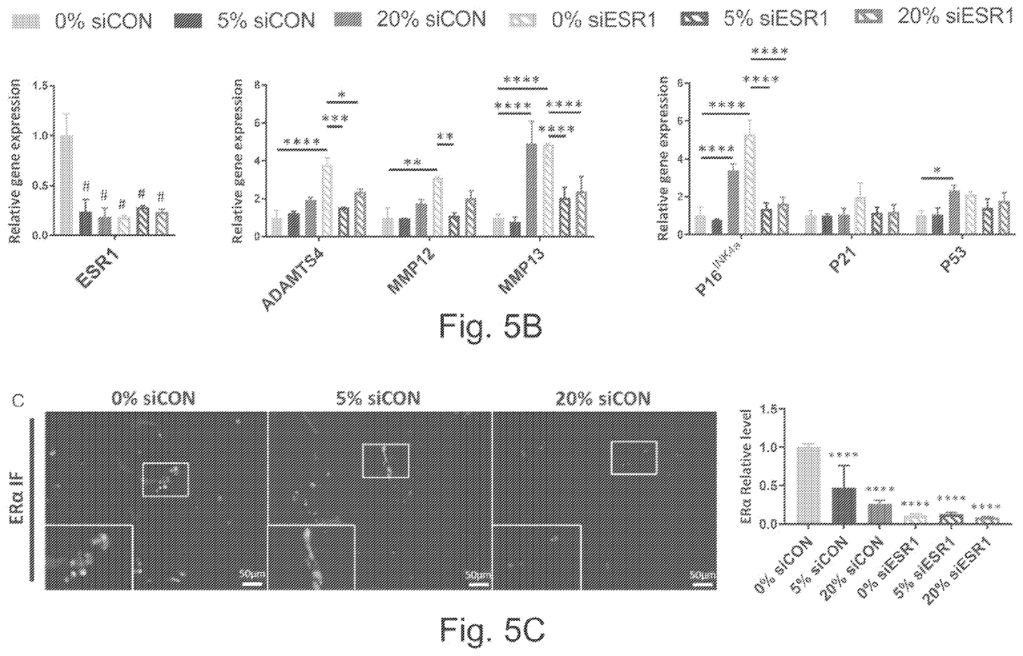

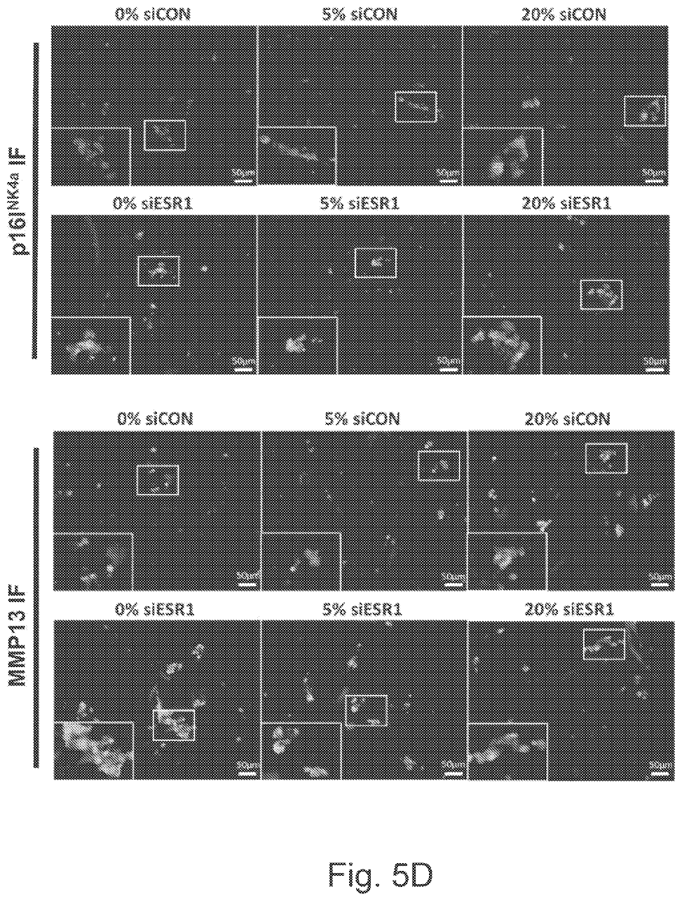

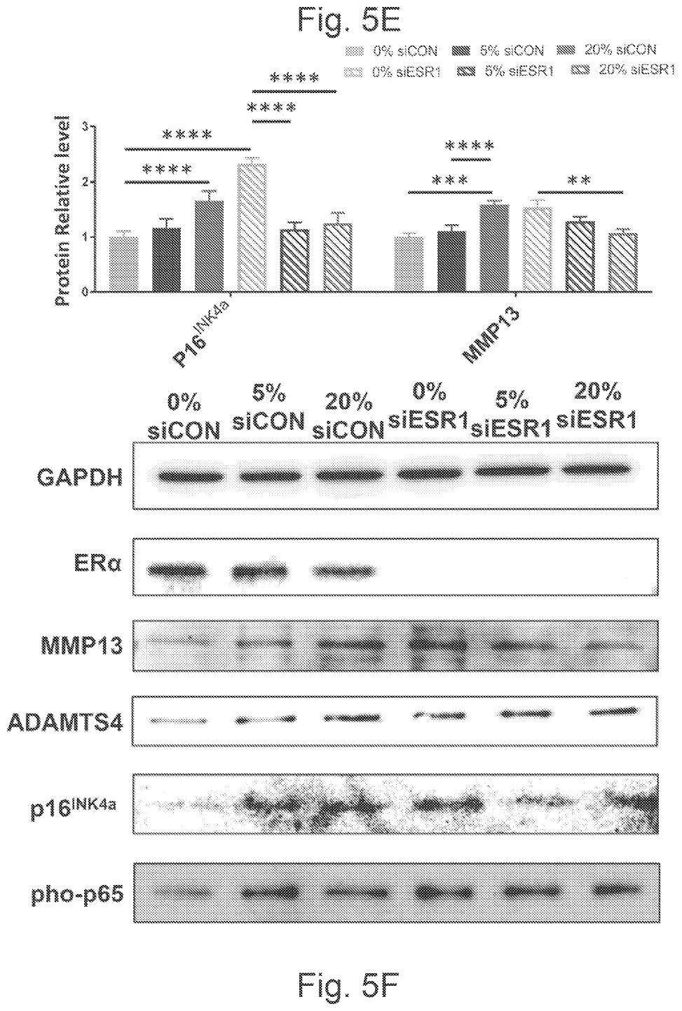

As D-C most often was found in areas of the knee associated with the highest in vivo mechanical stresses, it was further investigated whether reduced ERα level is associated with mechanical loading, and whether a reduced ERα level altered the chondrocyte response to mechanical loading. Since native chondrocytes reside in a 3D environment, P-CHs, treated with siCON or siESR1, were encapsulated into 3D gelatin-based scaffolds and then cultured in a Mechano-Active Tissue Engineering system (MATE) capable of compressing the cell-seeded constructs in a strain-controlled manner (represented schematically in FIG. 5A). In such studies, 0%, 5% and 20% strains were used, representing the situations of no loading, moderate loading and supraphysiological loading that chondrocyte may experience in vivo. As shown in FIGS. 5B, 5C and 5F, expression of ESR1 was decreased when P-CH-seeded constructs were subjected to dynamic compressive loading with 5% or 20% of strain. In addition, loading with 20% strain resulted in both upregulation of cartilage catabolic genes in siCON cells, such as ADAMTS4 and MMP13, and the upregulation of p16^{INK4A} and p53 (FIGS. 5B and 5D through 5F). These patterns were seen for both transcription (FIG. 5B) and translation (FIGS. 5D through 5F). These results indicate that mechanical overload, as modeled by supraphysiological 20% strain, was capable of producing an OA and senescent phenotype in chondrocytes.

Considering the demonstrated influence of ERα on chondrocyte senescence and the chondrogenic phenotype, and its responsiveness to mechanical loading, the role of ERα in modulating the effects of compressive loading on chondrocyte phenotype was investigated. Such studied were achieved by exposing chondrocyte-seeding gelatin constructs to three different loading conditions (0%, 5% and 20% strains), with or without concomitant knockdown of ESR1. siRNA was used to reduce the expression of ESR1 in P-CHs (siESR1 group), and results confirmed that ESR1 expression was effectively suppressed by siRNA treatment (FIG. 5B).

Mechanical loading at 20% strain no longer induced previously observed increases in p16^{INK4A} and MMP13 expression when ESR1 expression was preserved. Rather, mechanical loading applied on samples from siESR1 group reduced the expression of those two molecules (FIGS. 5B and SD through 5F). Further, both mechanical loading and ESR1 knockdown increased the level of phosphorylated p65 (pho-p65) in P-CHs, a representative marker of cellular stresses.

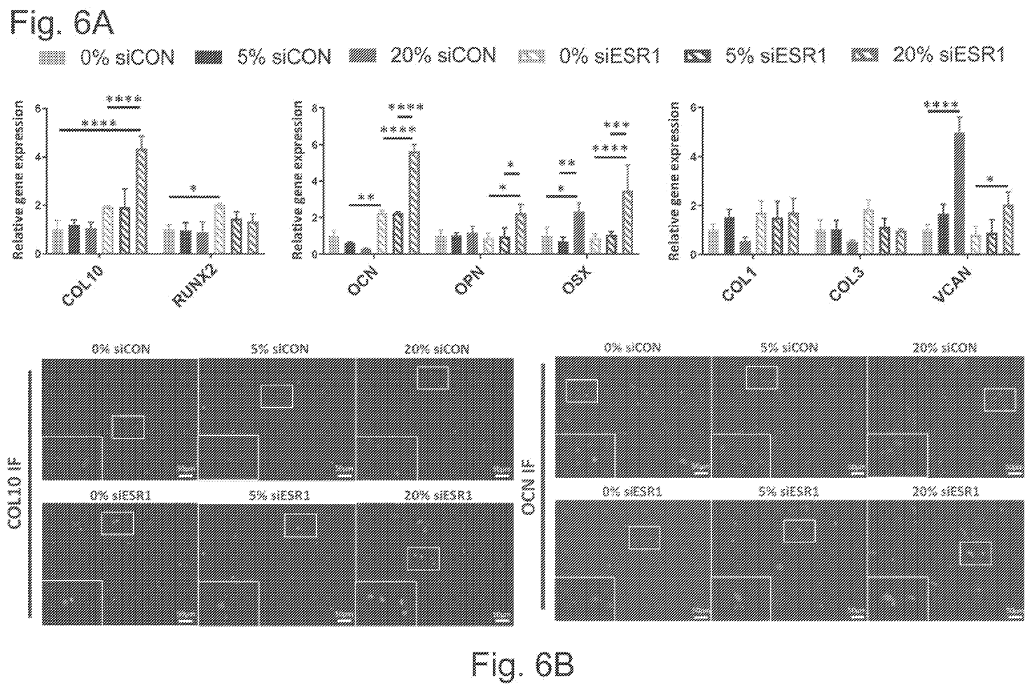

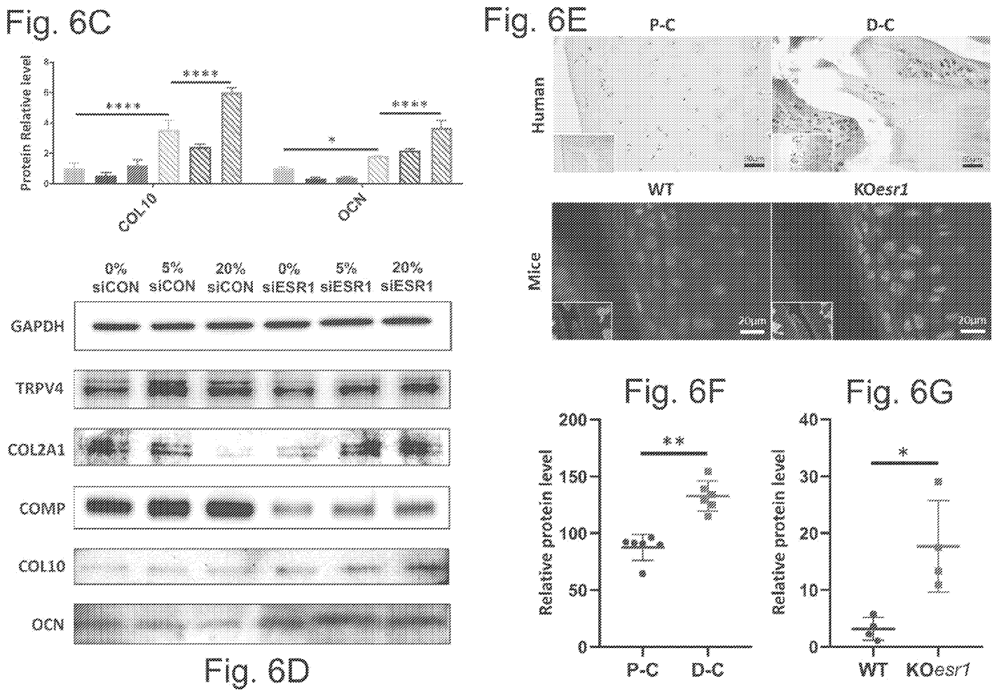

The interaction of mechanical loading and ERα expression in regulating chondrogenic, fibrogenic and osteogenic genes was also studied. Mechanical loading, at both 5% and 20% strain, did not significantly affect the expression of osteogenic (COL10, RUNX2, OCN, OPN) or fibrogenic (COL1, COL3) marker genes when ESR1 expression was preserved (siCON), although OSX and VCAN were significantly upregulated with 20% compressive strain (FIG. 6A). However, there was a strain-responsive downregulation of COL2A1 (FIG. 6D). Interestingly, ESR1 knockout alone (0% siESR1) remarkably upregulated the expression of OCN while suppressing levels of COL2A1 and cartilage oligomeric matrix protein (COMP) (FIGS. 6A through 6D). While 5% strain compressive loading had no mitigating effect on ESR1 knockout, 20% strain tended to further upregulate hypertrophy-associated markers, including COL10 and OCN (FIGS. 6B through 6D), while also reversing the inhibitory effect of ESR1 knockout on the levels of COL2A1 and COMP (FIG. 6D). Enhanced OCN level was also observed in D-CHs in humans when compared to P-CHs (FIGS. 6E and 6F), and in cartilage isolated from esr1 knockout mice when compared to the wild-type control (FIGS. 6E and 6G). The knockout of esr1 resulted in the loss of GAGs in cartilage in mice at age of 12 weeks but did not lead to other noticeable OA-relevant structural changes.

Expression of transient receptor potential cation channel subfamily v member 4 (TRPV4), a well-recognized mechanosensitive Ca²⁺ channel, was also responsive to mechanical loading. In the siCON group, loading at 5% strain significantly upregulated the expression of TRPV4. However, supraphysiological compressive strain (20%) modestly reduced TRPV4 expression when compared to 5% strain (FIG. 6D), No similar trend was observed in the siESR1 group. ESR1 knockout (0% siESR1) did not change TRPV4 protein levels but did modulate the effect of mechanical loading as increasing the strain induced a small but observable dose-dependent increase in TRPV4 expression (FIG. 6D).

The studies hereof described above identify a novel role of ERα in osteoarthritis and chondrocyte senescence. Notably, levels of ERα were significantly downregulated in severely damaged cartilage (D-C) of osteoarthritic knees that underwent TKA, as compared to regions of preserved cartilage (P-C) of the same knee, Given the shared genetic and biochemical environment of D-C and P-C located within the same knee, the differences in tissue integrity and resulting chondrocyte phenotype could, in part, be attributed to differences in the mechanical microenvironment. ERα has not been previously shown to possess a similar role in chondrocytes. Using a 3D gelatin-based hydrogel embedded with chondrocytes to which cyclic compressive loading was applied to model physiologic (5%) and injurious (20%) strains, it was shown that injurious strains downregulated ERα with resulting loss of chondrocytic phenotype but increased expression of inflammation and degradation-associated molecules. In addition, knockdown of ERα changed chondrocyte responsiveness to mechanical loading by further upregulating hypertrophic and osteogenic markers. Furthermore, ERα appeared to be upstream of senescent markers, including p16^{INK4A}. Accordingly, knockdown of p16^{INK4A} did not reverse the osteoarthritic phenotype, while restoration of ERα led to the downregulation of senescence marker expression and partially reversed the osteoarthritic phenotype.

Chondrocyte senescence is present in osteoarthritic cartilage, yet the role of senescence in disease onset and progression remains debatable. Specifically, studies have shown that chondrocytes isolated from damaged cartilage expressed typical senescence markers, such as high levels of p16^{INK4A} and SA-β-Gal, with increased production of senescence-associated secretory phenotype (SASP) factors. Similar results were observed in the studies hereof. However, whether these phenotypic changes constitute senescence, entailing a permanent loss of cell proliferative capacity, was still uncertain. OA chondrocytes, even those isolated from severely damaged cartilage (D-C), were found to still possessed the ability to proliferate in the present studies, although the replication capacity of D-CHs was lower than P-CHs. Similarly, it has been demonstrated that the effects of chondrocyte senescence on OA are more likely driven by the production of SASP molecules rather than by a loss of chondrocyte replicative function. Furthermore, based on the present studies, the expression of senescent markers and impaired chondrocyte function can be at least partially reversed through enhancing ERα expression. Therefore, it was hypothesized that chondrocytes in D-C are not senescent in the formal definition, but rather have an impaired chondrogenic phenotype amenable to therapy. It is surmised that the cells in damaged cartilage possess a senescent phenotype as a result of their microenvironment rather than intrinsic cell processes. To that end, once the OA-relevant stresses are removed or reduced, these chondrocytes may revert to a healthier state, as demonstrated in the studies hereof.

In view of the results of the studies hereof, a microenvironment-centric view of chondrocyte senescence in OA is indirectly supported through a number of other recent observations. Selective removal of senescent chondrocytes in the OA joint by senolytic agents was shown to alleviate OA severity in a murine model, which supports the adverse effects of SASP mediators in promoting or maintaining the senescent phenotype in OA pathogenesis. However, maintenance of cartilage integrity relies on chondrocyte homeostasis, with targeted removal of chondrocytes potentially further compromising the prospect of endogenous cartilage repair. If OA chondrocytes are temporarily in a “senescent-like” state that can be reversed, irreversible chondrocyte destruction may not be warranted. What may at first provide apparent improvement in cartilage integrity may ultimately obviate cartilage repair. For example, intra-articular injection of senolytic ABT-263 in a phase II clinical trial found that ABT-263 failed to outperform a placebo in the reduction of joint pain and stiffness in patients with knee OA. As an alternative to senolytics, senomorphics may mitigate the detrimental influence of senescent cells without irreversible chondrocyte removal. Instead of killing senescent cells (the function of senolytics), senomorphics function by reducing senescent marker expression and the production of SASP molecules. Studies hereof demonstrate that restoring ERα can reverse the senescence level, indicated by reduced p16^{INK4A} and the expression of SASP factors.

In addition to consideration of the potential role of senolytic or senomorphic therapy for chondrocyte senescence, the causative role of senescent marker p16^{INK4A} is not fully understood. The protein p16^{INK4A} has many different roles in numerous biological processes, and its expression is regulated by different factors, such as cellular stress. The results hereof indicate that mechanical loading enhanced the level of p16^{INK4A}, even under physiological strains (FIG. 5F). Nevertheless, suppression of p16^{INK4A} did not directly reverse the osteoarthritic phenotypes in D-CHs (FIG. 3), again suggesting that p16^{INK4A} is a marker, but not the driver, of chondrocyte senescence in OA. Indeed, another in vivo study demonstrated that inactivation of p16^{INK4A} in chondrocytes of adult mice did not mitigate SASP expression or alter the rate of osteoarthritis (OA) induced with physiological aging or after destabilization of the medial meniscus.

As direct suppression of p16^{INK4A} did not reverse the osteoarthritis phenotype, the upstream molecules that regulate both p16^{INK4A} and other OA-relevant molecules were investigated. Through RNA-Seq, expression of ESR1 gene was found to be downregulated in D-CHs, which was additionally confirmed by RNA-Seq data from two previous studies with publicly open data sets of large sample sizes from GEO (GSE57218, E-MTAB-4304). The functional consequence of a ERα in OA chondrocytes had not previously been determined. Similar to human samples, the studies hereof also demonstrated for the first time that ERα was also downregulated in aged mice with OA. It is clear that ERα reduction accompanies OA progression. ERα is conventionally recognized as a nuclear receptor of estrogen, which has been shown to function through a ligand-dependent mechanism as expected in articular cartilage chondrocytes and growth plate chondrocytes. However, as high levels of ERα were found in preserved cartilage from both male and female donors, it was hypothesized that ERα may also function in a ligand-independent (i.e., mechanoresponsive) manner. Such a non-conventional mechanism of ERα has never been proposed in cartilage.

As described above, the relevance of ERα and mechanical loading in chondrocytes was investigated in studies hereof, and results hereof showed that the ERα level was regulated by compressive loading, which further mediated cellular responsiveness to mechanical cues. Supraphysiological compressive strains (20%) downregulated ERα expression with concomitant loss of chondrogenic phenotype and up-regulated hypertrophic marker expression. Targeted reduction in ERα with siRNA further resulted in the synthesis of COL10 and osteocalcin, highlighting the role of ERα in maintaining the phenotype of chondrocytes. Chondrocytes with low ERα level converted all mechanical cues, at either physiologic or injurious strains, into upregulated expression of cartilage ECM molecules. This outwardly contradictory observation may imply a self-reparative mechanism to prevent early bone-to-bone contact in OA pathogenesis. In fact, in the D-C, the mixed expression of cartilage and bone markers was observed. Therefore, the reduction of ERα results in the acquisition of both senescent and osteoarthritic phenotypes in chondrocytes, with resulting hypertrophic cartilage-like reparative response upon further mechanical stimulation.

Once again, the above-discussed studies hereof demonstrate that estrogen receptor a (ERα), which is a member of the steroid/nuclear receptor family, plays a role in maintaining the chondrocyte phenotype. Further, knock-out of ESR1, the gene encoding ERα, results in the generation of a senescent phenotype in chondrocytes isolated from intact cartilage, while knock-in of ESR1 reduces the senescence level of chondrocytes isolated from severely damaged cartilage. The newly identified function of ERα in reducing senescence of chondrocytes was observed in cells from both male and female donors and did not need the presence of ligands, such as estradiol. The reason for the loss of ERα in OA chondrocytes is not apparent from the studies discussed above. In breast cancer cells, it has been shown that deficiency of DNA damage repair is associated with the loss of ERα, and a function of ERα in regulating the DNA damage response (DDR) pathway was reported, suggesting their bidirectional interaction. It was hypothesized that DNA damage resulted in the reduction of ERα levels in chondrocytes, which in turn led to the generation of the senescence phenotype.

To evaluate that hypothesis, the levels of DNA damage and senescence in normal and OA cartilage samples from human donors and mice were determined and correlated to the expression levels of ERα. Human chondrocytes isolated from healthy articular cartilage were treated with doxorubicin (DOX) to induce DNA damage. Results showed that DOX-induced DNA damage was accompanied by the reduction of ERα levels. Knock-in of ESR1 reduced the level of DNA damage and cellular senescence in chondrocytes. Furthermore, it was discovered that the senescence-suppressing function of ERα was achieved partially by inhibiting the NF-κB pathway. The studies hereof further indicate the potential of restoring or maintaining ERα levels in treating or preventing OA. The studies hereof related to DNA damage are sometimes referred to herein as DNA-related studies.

In the DNA-related studies hereof, the ERα levels in normal and OA cartilage were first compared using samples from both mice and human donors. Severe degradation and loss of GAGs were observed in cartilage undergoing destabilization of medial meniscus surgery (DMM) in a mouse model when compared to the sham control. In general, a sham surgery omits the step thought to be therapeutically necessary. The DMM surgical model has become a gold standard for studying the onset and progression of posttraumatic osteoarthritis. In that regard, the DMM model mimics clinical meniscal injury, which is a known predisposing factor for the development of human OA. DMM permits the study of structural and biological changes over the course of the disease. The OA phenotype was also accompanied by reduced ERα levels. Similar findings were observed in human samples. In the cartilage harvested from donors with OA, ERα levels in the surface area were lower than that in the healthy counterparts.

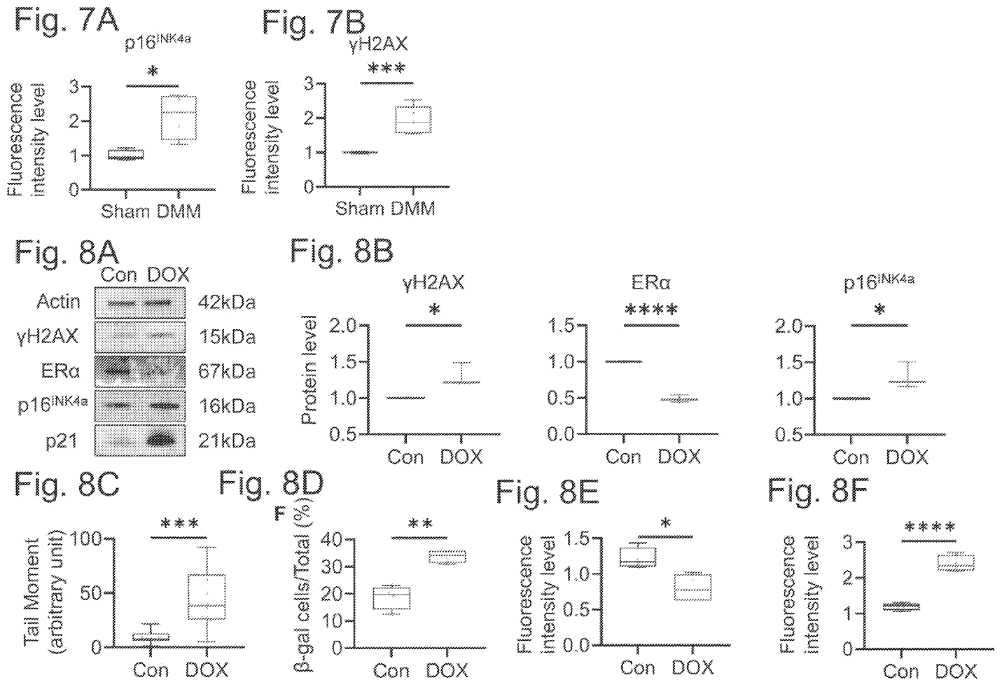

Subsequently, the p16^{INK4A} levels in cartilage harvested from animals undergoing Sham or DMM was examined. Results showed a significant upregulation of p16^{INK4A} in the DMM group (FIGS. 7A and 7B), implying a senescent phenotype in OA cartilage.

DNA damage is a common cause leading to senescence. The level of γH2AX, a representative marker of DNA damage was thus analyzed. As shown in FIGS. 7C and 7D, a significantly increased γH2AX level was observed in the samples from DMM mice. In human samples, low ERα levels were correlated with the increased level of p21. Moreover, suppressing ERα with siRNA resulted in the downregulation of RAD50, which is a crucial molecule for sensing and repair of DNA damage. Taken together, OA chondrocytes contain DNA damage and deficiency of DNA repair. Moreover, OA chondrocytes display senescence features, which may be associated with reduced ERα levels.

The results discussed above suggest that DNA damage might suppress ERα expression, thus inducing a senescent phenotype in chondrocytes. To study that hypothesis, doxorubicin (DOX) was introduced into the chondrocyte culture to induce DNA damage. In such studies, healthy chondrocytes harvested from 4 male and 4 female donors were used. After 3 days of treatment of DOX, significant DNA damage was observed, which was revealed by increased levels of p21, γH2AX (FIGS. 8A and 8B), and the comet assay (FIGS. 8C and 8D). As known in the medical and biological arts, the comet assay is a single cell gel electrophoresis assay which provides a sensitive techniques for detecting DNA damage. The generation of senescent phenotype was observed after DOX treatment, which was confirmed by the increased SA-β-Gal staining (FIGS. 8E and 8F) and the upregulation of p16^{INK4A} and p21 (FIGS. 8A, 8B, 8G and 8H). Importantly, the ERα level was significantly reduced after DOX treatment (FIGS. 8A, 8B, 8I, and 8J). These results collectively indicated that DOX treatment resulted in DNA damage, senescence, and the loss of ERα in human chondrocytes.

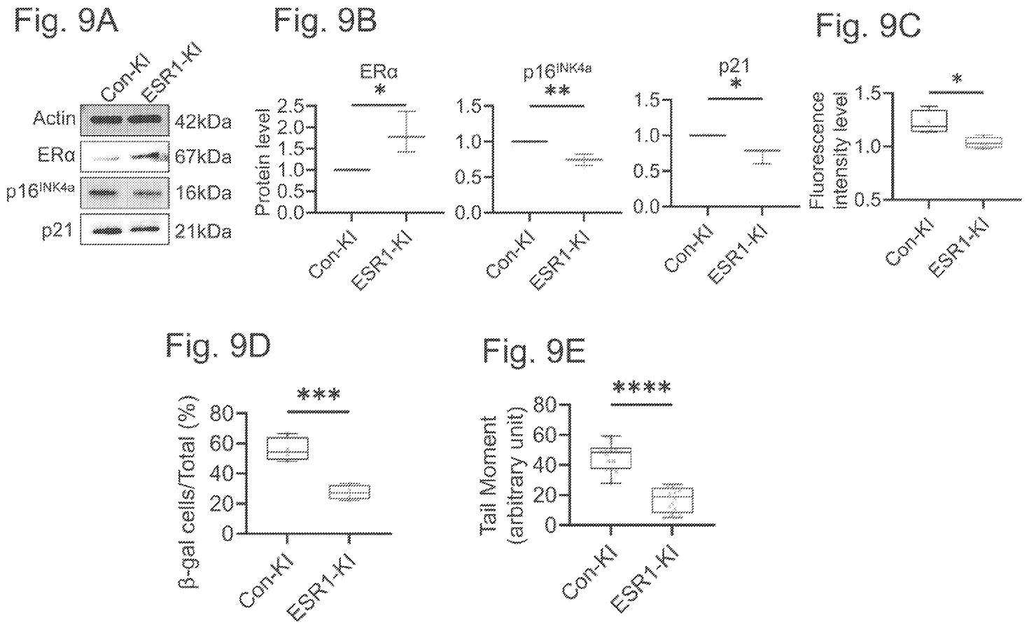

To examine if maintaining ERα could suppress DNA damage-induced senescence, lentiviral vectors were used to deliver the ESR1 gene into human chondrocytes that had been pre-treated with DOX. The expression of ESR1 was driven by a constitutive promoter. Three days after transduction, cells carrying control gene mCherry (CON-KI group) or the ESR1 (ESR1-KI group) gene were analyzed. The success of overexpressing ESR1 was confirmed by western blot (FIGS. 9A and 9B). Overexpression of ESR1 in DOX-treated chondrocytes significantly suppressed p16^{INK4A} and p21 levels (FIGS. 9A, 9B, 9C, and 9D), as well as reduced the number of SA-β-Gal-positive cells from 56% to 27% (FIGS. 9E and 9F) in DOX-pretreated chondrocytes. Knock-in of ESR partially reduced the level of DNA damage (FIGS. 9G and 9H) as revealed by the comet assay.

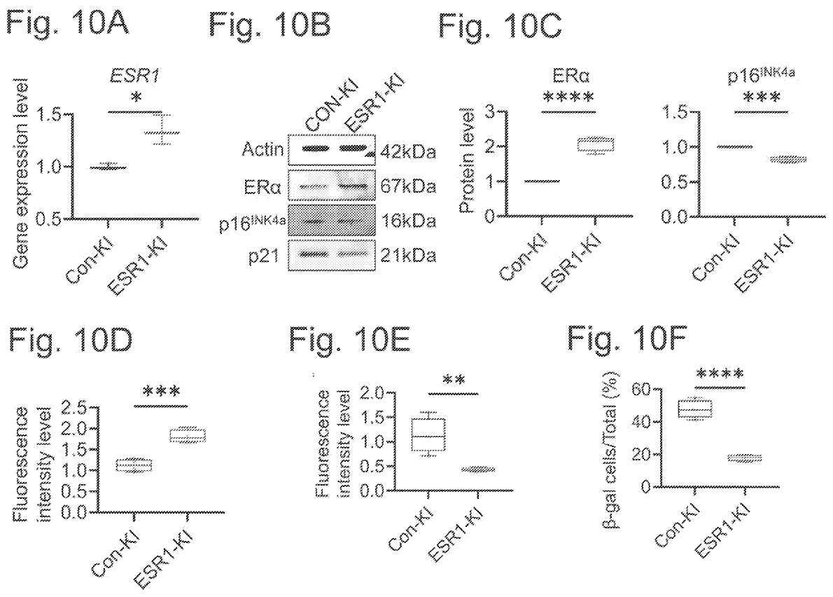

The therapeutic potential of overexpressing ESR1 was examined using human chondrocytes from 3 male and 3 female donors. Specifically, OA chondrocytes were treated with vectors carrying the control gene mcherry (CON-KI group) or ESR1 (ESR1-KI group). The success of overexpressing ESR1 was confirmed by real-time qPCR (FIG. 10A), western blot (FIGS. 10B and 10C), and immunostaining (FIGS. 10D and 10E). In the cells from the ESR1-KI group, the ratio of SA-β-Gal positive cells (FIGS. 10F and 10G), p16^{INK4A} and p21 levels (FIGS. 10B, 10C, 10H, and 10I), were significantly lower than those from the CON-KI group. The results indicated that overexpression of ESR1 suppressed senescent phenotype in OA chondrocytes.

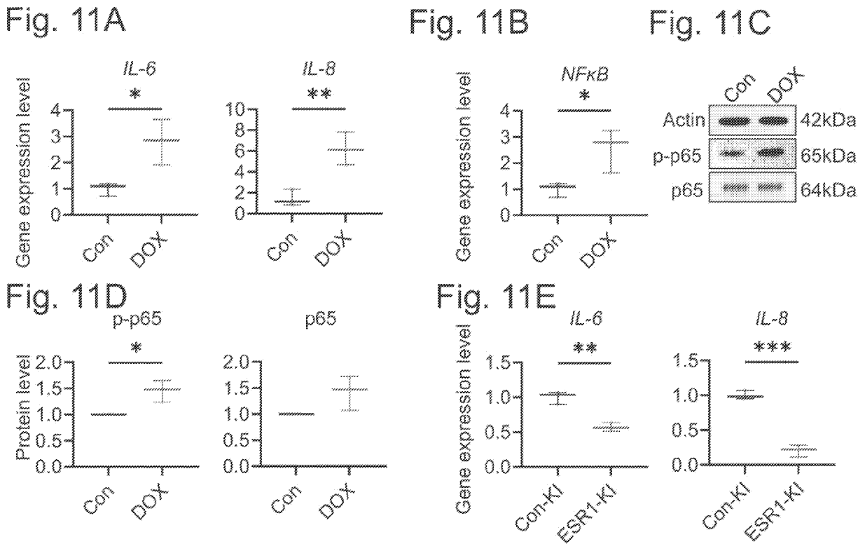

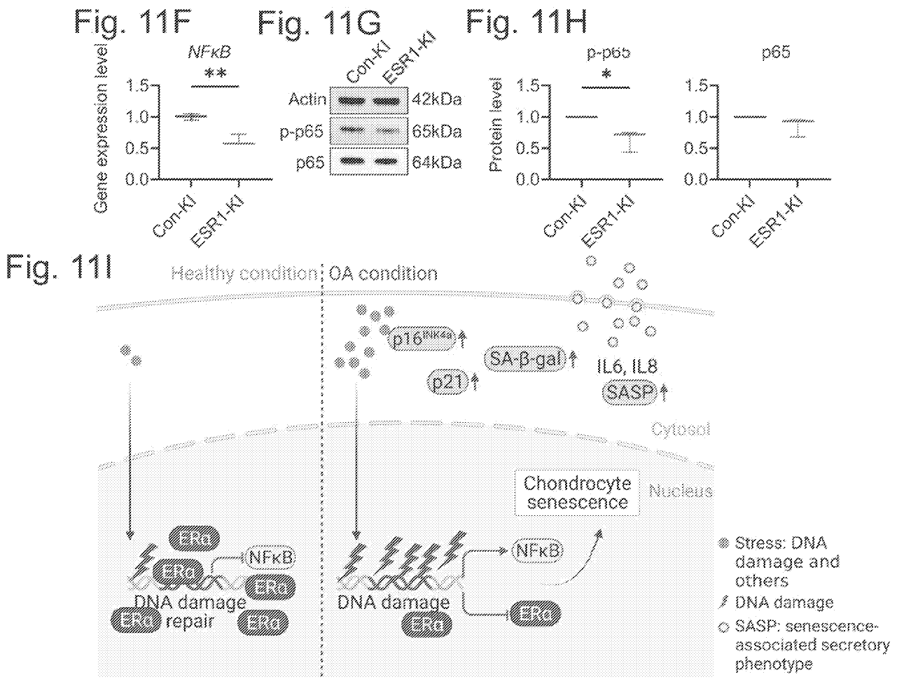

FIGS. 11A through 11I illustrates a potential role of ERα in regulating NF-κB pathway. In the studies of FIGS. 11A through 11D, normal human chondrocytes were treated with DOX (100 nM, DOX group) or vehicle control (Con group) for three days. In the studies of FIGS. 11E through 11H, normal human chondrocytes were pre-treated with DOX for three days and then transfected with vectors carrying mCherry (CON-KI group) or ESR1 (ESR1-KI group) gene. In FIGS. 11A and 11E, relative gene expression levels of IL6 and IL8 were examined by qPCR. The data are normalized to the respective Con (A) or CON-KI (E) group, respectively. In FIGS. 11B and 11F, relative gene expression level of NI-κB. The data are normalized to the respective Con (B) or CON-KI (F) group, respectively. In FIGS. 11C and 11G, protein level of Actin, phosphorylated p65 (p-p65), and total p65 were examined by western blot. In FIGS. 11D and 11H, semi-quantification of p65 and p-p65 levels based on the band intensities from the western blot shown in FIGS. 11C and FIG. 11G, respectively. The data are normalized to the Con (D) or CON-KI (H) group, respectively. In FIGS. 11A through 11H, *, p<0.05; **, p<0.01; ***, and p<0.001.

The mechanism underneath ERα suppressing senescence was not clear. Results from real-time qPCR indicated that DOX resulted in the upregulation of interleukin-6 (IL-6) and interleukin-8 (IL-8)(FIG. 11A). DOX-induced increase of IL-6 and IL-8 expression levels were partially reversed by overexpression of ESR1 (FIG. 11E). The expression of pro-inflammatory cytokines is often regulated by the NF-κB pathway. See, for example, Lawrence, T., (2009) The nuclear factor NF-kappaB pathway in inflammation. Cold Spring Harb Perspect Biol, a001651. In addition, senescence has been shown to be associated with the activation of the NF-κB pathway. It was examined if ERα functioned through regulating NF-κB pathway. Specifically, the changes of p65, a key molecule in the NF-κB pathway, were measured with western blot. As shown in FIGS. 11B through 11D and FIGS. 11F through 11H, DOX treatment activated the NF-κB pathway, indicated by the upregulation of NFκB gene and activation of p-p65. Knock-in of ESR1 significantly suppressed NFκB expression and p-p65 level (FIGS. 11F through 11H), suggesting that ERα functions, at least partially, through suppressing the NF-κB pathway. Without limitation to any mechanism, the interactions of ERα with DNA damage and cellular senescence-associated molecules are proposed in FIG. 11I. In healthy chondrocytes, the normal level of ERα suppresses DNA damage and NF-κB pathway. Under OA conditions, with insults from different stressors, such as DNA damage, the ERα level was reduced, which resulted in the activation of NF-κB and the establishment of senescence phenotype. In addition to the NF-κB pathway, other potential pathways may be involved.

The above-described DNA-related studies on animal and human samples demonstrated that chondrocytes in OA cartilage display a higher degree of DNA damage and senescence, accompanied by the reduction of ERα levels when compared to cells in healthy cartilage. DNA damaging agent DOX resulted in the generation of senescence in cultured human chondrocytes, which also suppressed the expression of ERα. In addition, overexpression of ERα was able to reduce the levels of DNA damage and the resultant senescence. Lastly, ERα suppressed DOX-induced activation of p65, thus reducing the expression of pro-inflammatory cytokines. The results hereof collectively indicated the critical role of ERα in regulating the cellular response to DNA damaging signals and suppressing the generation of senescent phenotype in chondrocytes.

A high ratio of senescent cells has been found in OA cartilage samples collected from animal models and humans. Although the exact mechanism is not clear, it has been proposed that accumulated damages due to different types of stressors lead to the generation of senescence phenotype. In addition to well-studied reactive oxygen species (ROS) and pro-inflammatory cytokines, results hereof have showed that supraphysiological mechanics also induced chondrocyte senescence. Regarding the roles of senescence chondrocytes in OA pathogenesis, it is still not clear whether they are just one of the consequences of injuries or a key driver in accelerating the progression of OA. As described above, it has been shown that selectively eliminating senescent cells in knee joint attenuated post-traumatic OA in rodents. However, the exciting finding in the preclinical study was not able to translate into successful therapy. In fact, the relevant human clinical trial was terminated in Phase I. Therefore, killing senescent cells as an OA treatment method requires further investigation. However, the inferior state associated with chondrocyte senescence seems to be reversible. Therefore, instead of killing these senescent cells, an alternative strategy to ease the burden of senescence is to use specific compounds to reduce the level of senescence, which is so-called senomorphics. In any event, it is valuable to understand the transition and maintenance of chondrocyte senescence to identify targets that can be used to treat OA.

DNA damage is probably the most studied mechanism causing cellular senescence. Oxidative stress and replicative stress can, for example, both induce DNA damage. Through in vitro experiments, previous reports have shown that stochastic genomic DNA damage induced by increased oxidative or genotoxic stress induced the heterogeneity in gene expression found in the OA cells in situ. Irradiation has been used to induce DNA damage in healthy cartilage explants, and results showed chondrocytes within explants emerged with persistent DNA damage response increased p16^{INK4a} and SA-β-gal activity. Such studies revealed that accumulated DNA damage and subsequent chaotic gene activation pattern in chondrocytes is an important pathological change in OA. Recently, another study demonstrated accumulated DNA damage in aged and OA chondrocytes by using comet assay, which highlighted the potential for DNA damage to contribute to chondrocyte senescence and OA pathogenesis. Previous studies had also showed that several DNA damage-responding molecules and associated pathways are involved in OA progression. For example, is has been discovered that the deficiency of regulation in development and DNA damage response 1 (REDD1) increased the severity of changes in cartilage, menisci, subchondral bone, and synovium in the DMM model of OA. Sinuin 6 (SIRT6) is a member of the sirtuin family of nicotinamide adenine dinucleotide (NAD(+))-dependent protein deacetylases, and depleting SIRT6 in human chondrocytes caused increased DNA damage and subsequent premature senescence. Targeting DNA damage in chondrocytes may represent a new therapeutic strategy for the treatment of OA.

H2AX belongs to the H2A histone family that facilitates the organization of chromatin. When the nuclear double-stranded DNA breaks, H2AX protein is phosphorylated by ATM/ATR at position Ser139 to form γH2AX. ATM (ataxia-telangiectasia mutated), ATR (ATM- and Rad3-Related), and DNA-PKcs (DNA-dependent protein kinase) kinases are the most upstream DDR kinases. The expression of γH2AX could thus reflect DNA damage levels. In studies hereof, the immunostaining clearly demonstrated the high level of γH2AX in cartilage from DMM animals. Previous studies have shown that the level of γH2AX was increased in both DMM mice model and cartilage explants under irradiation and mitogenic stimulation. The results together confirmed the presence of DNA damage in OA chondrocytes.

Currently, the most established factors that mediate the connection between DNA damage and senescence are p21/p53 and p16^{INK4a}. In OA chondrocytes, those three proteins are also highly upregulated, which was found in the present studies. The studies hereof showed that inhibiting ERα resulted in increased p16^{INK4a} levels and the generation of senescence phenotype in chondrocytes and that overexpressing ERα suppressed the expression of p16^{INK4a} and senescence levels. The DNA-related studies hereof, further examined whether ERα also regulates p21. The results hereof indicated that knock-in of ESR1 decreased p21 levels in OA chondrocytes. Therefore, overexpressing of ESR1 could at least partially reverse the senescence phenotype in DOX pre-treated chondrocytes. Such findings again highlight that ERα is a strong anti-senescence factor. Results indicate the potential of restoring ERα level in reducing senescence level. From the therapeutic perspective, a small molecule-based treatment would be ideal, given that the level of safety of injecting viral vectors has not been fully determined in humans. Decitabine (DAC, 5-Aza-2′-deoxycytidine) may, for example, increase ERα levels in osteosarcoma cells. The nucleoside analog decitabine is a cytidine analog. 4-hydroxytamoxifen may also be used to increase ERα levels in osteosarcoma cells. Other selective estrogen receptor modulators are known and include, for example, Raloxifene, Ospemifene, and Bazedoxifene.

Results hereof indicate the involvement of ERα in the cellular response to DNA damage. FIGS. 10A through 10I provide direct evidence that knock-in of ESR1 attenuated DNA damage. ERα may directly reduce DNA damage and mitigate the resultant senescence in somatic cells, including chondrocytes. Studies hereof showed that supraphysiological mechanical loading, not physiological stimulation, also reduced ERα. These discoveries collectively led to the hypothesis that ERα is a hub that may mediate stressors-induced senescence. The finding is also informative to the hormone replacement therapy (HT) for menopausal arthritis. Although it has been previously shown that estrogen provides protective effects on chondrocytes, the therapeutic effect of estrogen replacement therapy on human OA is still inconclusive.

A salient characteristic of chondrocytes senescence is the senescent-associated secretory phenotypes (SASPs). Pro-inflammatory cytokines, such as tumor necrosis factor-α (TNF-α), interleukin-1β (IL-1β), IL-6, IL-8, are important components of SASPs, which can induce low-grade inflammation and cartilage degradation in peripheral joint tissues. In addition, it has been reported that prolonged exposure to IL-6 and IL-8 promoted cellular senescence, suggesting that inflammation can induce a cross-reinforced senescence milieu response. DOX treatment has been shown to directly stimulated inflammation in chondrocytes. Therefore, the expression levels of two representative pro-inflammatory cytokines, IL-6 and IL-8, was examined under different conditions. DOX treatment elevated the expression of IL6 and IL8, which was partially reversed through overexpression of ERα. To explore the underlying mechanism, the level of p65, a key component in the NF-κB pathway, was examined and the results clearly indicated that phosphorylation of p65 in chondrocytes was suppressed when overexpressing ERα. That conclusion was supported by the other studies which showed that ERα could tether with other transcription factors (TFs) and influence the NF-κB pathway. It has also been shown that NF-κB was activated in chondrocytes of knee joints from OA mice. The NF-κB suppressing function of ERα again highlighted its role in maintaining the health of cartilage. The functions of ERα defined in the present study were based on cell culture in a serum- and phenol red-free medium without the supplementation of ligands.

The studies hereof thus demonstrate that the OA chondrocytes from mice and human donors contained DNA damage and displayed a senescence phenotype. This was accompanied by significantly reduced ERα levels. Overexpressing/increasing the expression of ERα not only reduced the senescence levels in OA or doxorubicin-treated chondrocytes but also suppressed their senescence phenotype. Mechanistically, ERα inhibited the activation of NF-κB pathway, thus reducing the production of the senescence-associated secretory phenotype (SASP) factors. These findings provide insights into dynamic signaling pathways regulating DNA damage and senescence in chondrocytes, thereby advancing the understanding of the role of cellular senescence in OA pathogenesis. Once again, the studies hereof indicate that maintaining the ERα levels represents a new avenue to prevent and treat OA.

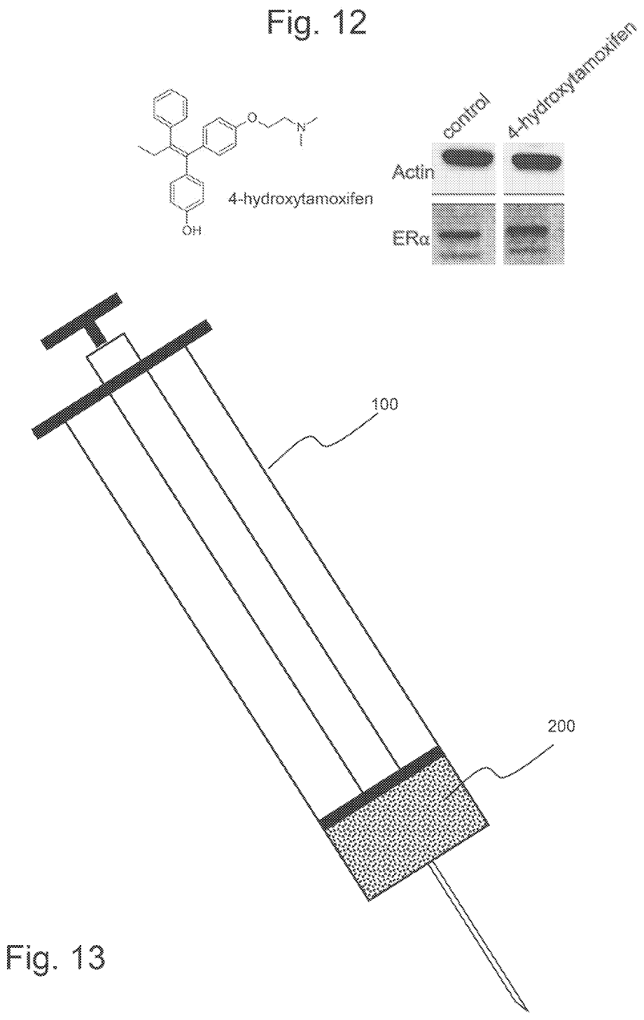

In a number of embodiments, systems, methods, and composition hereof provide for treatment of osteoarthritis by increasing estrogen receptor-α in affected cartilage. For example, estrogen receptor-α may be increased via delivery of at least one of an agent to effect knock-in of an estrogen receptor-α gene, an agent to effect interference with microRNA which suppress estrogen receptor-α gene expression, or an agent that enhances estrogen receptor-α gene expression. An agent to effect knock-in of an estrogen receptor-α gene may, for example, include a plasmid DNA comprising an estrogen receptor-α gene. The agent to effect knock-in of an estrogen receptor-α gene may further include one or more transduction agents. An agent to effect interference with microRNA which suppress estrogen receptor-α gene expression may, for example, include siRNA. An agent that enhances estrogen receptor-α gene expression may, for example, include or be a small molecule compound. The agent that enhances estrogen receptor-α gene expression may, for example, be a peptide or a selective estrogen receptor modulator. The selective estrogen receptor modulator may, for example, be or include 4-hydroxytamoxifen. In a number of studies with 4-hydroxytamoxifen, human chondrocytes were cultured in the wells of 6-well plate and then treated with 1 uM 4-hydroxytamoxifen or DMSO control for 48 hours. Afterward, western blot was used to examine ERα level. Actin was used as the loading control. The results, which are illustrated in FIG. 12, indicate that 4-hydroxytamoxifen upregulate the level of ERα.

Typically, an agent to increase estrogen receptor-α in affected cartilage (for example, at least one of the agent to effect knock-in of an estrogen receptor-α gene, the agent to effect interference with microRNA which suppress estrogen receptor-α gene expression, or the agent that enhances estrogen receptor-α gene expression) is delivered locally to the affected cartilage (for example, by injection). FIG. 13 illustrates schematically a delivery system such as an injector system 100 (which may be manually operated or powered) to deliver an agent 200 to increase estrogen receptor-α in affected cartilage.

Small-molecule compounds and other treatments may, for example, be intraarticularly injected in solution, which is a simple and straightforward strategy to apply drugs to influence chondrocytes, in pharmaceutically effective amounts. Alternatively, clinical and preclinical studies suggest that sustained and controlled drug release, such as through the polymeric implantable drug delivery systems, can avoid complications due to repeated injections, such as infection and unnecessary systemic exposure to high doses of drugs. Therefore, one can encapsulate small-molecule compounds and/or other treatments hereof into a carrier, such as polymer-based (for example, poly(lactic-co-glycolic acid) (PLGA)) microparticles or nanoparticles, which can provide long-term and controlled release through only one intraarticular injection. For example, 1-5 mg of compounds can be loaded into 3-15 mg microparticles, which will be intraarticularly injected based on 1-5 mg compounds/1 kg body weight.

Small-molecules compounds may be administered in a pharmaceutically effective amount of a compound, a pharmaceutically acceptable salt of the compound or a pharmaceutically effective prodrug. In general, treatments for increasing estrogen receptor-α in affected cartilage may be administered by any conventional route of localized administration. In general, a pharmaceutically effective amount or dosage contains an amount of one of the treatment effective to increasing estrogen receptor-α and display anti-OA behavior. Pharmaceutical compositions containing as an active ingredient to increase estrogen receptor-α, a pharmaceutically acceptable salt thereof, or a prodrug in association with a pharmaceutically acceptable carrier or diluent are also within the scope hereof. In general, treatments hereof for increasing estrogen receptor-α may be constituted into any form suitable for the mode of administration.

### Experimental

Harvest of preserved cartilage (P-C) and damaged cartilage (D-C) from the same knee joint. With approval from the institutional review boards (IRB) of the University of Pittsburgh and University of Washington, human OA knee cartilage tissues were collected from patients who underwent TKA (Total knee joint arthroplasty). Orthopedic surgeons with expertise in assessing human knee cartilage lesions reviewed all samples. P-C and D-C were first distinguished by Outerbridge scoring (FIG. 1A). Tissue samples (1 cm diameter) with homogenous macroscopic appearance were core-reamed from the articular surface using a rotatory cutter (Dremel, Racine, WI, USA) and distinguished according to an Outerbridge score ≤1 or ≥2, defining P-C and D-C, respectively. Next, a 4 mm diameter osteochondral cylinder was reamed from the center of the larger tissue sample, on which OA histopathology evaluation was performed. The remaining cartilage tissue (i.e., 1 cm diameter tissue sample without 4 mm diameter core) was harvested, from which chondrocyte isolation was performed. Cold PBS was used to avoid thermal damage to the osteochondral tissues during sample collection.

Isolation and Expansion of Human Chondrocyte from healthy and OA cartilage. Healthy human knee cartilage was collected from arthritis-free donors (Oversight of Research and Clinical Training Involving Decedents (CORID) approval by the University of Pittsburgh), and human OA cartilage was collected from patients who underwent total knee arthroplasty (Institutional Review Boards (IRB) approval by the University of Pittsburgh and University of Washington). The isolation methods have been reported in our previous studies. To assess the severity of cartilage degradation, OA histopathology was performed before cell isolation. Damaged OA cartilage was defined by Outerbridge scoring (Outerbridge score ≥2).

Isolated chondrocytes were expanded in Dulbecco's Modified Eagle Medium (DMEM, high glucose, Gibco/Thermo Fisher Scientific, Waltham, MA, United States) containing 10% fetal bovine serum (FBS, Life Technologies, Carlsbad, CA, United States) and 1% Antibiotic-Antimycotic (Life Technologies). Upon reaching 70-80% confluency, cells were detached by trypsin-0.25% ethylenediaminetetraacetic acid (EDTA, Thermo Fisher Scientific) and passaged. Chondrocytes derived from articular cartilage have limited in vitro proliferative potential (16). To minimize donor-to-donor differences and obtain sufficient cell numbers for this study, P2 healthy chondrocytes pooled from 8 donors (half male and half female), and P2 OA chondrocytes pooled from 6 donors (half male and half female), were used in this study. In the DNA-related studies hereof, all in vitro experiments were conducted in serum and phenol-red free medium after chondrocyte proliferation.

Histology (Safranin O/Fast green). Samples were fixed overnight at 4° C. in 10% buffered formalin (Fisher Chemical, Hampton, NH), decalcified in formic acid bone decalcifier (StatLab, Mckinney, TX, USA) for 2 days, dehydrated in 10% (w/v) sucrose, 20% sucrose and 30% sucrose for 1 hour each, embedded with Cryo-Gel (LeicaBiosystems, Richmond, IL, USA) and finally cryosectioned at 10 μm thickness with the use of Leica® CM1850 Cryostat (Mercedes Scientific, Lakewood Ranch, FL, USA). Samples were then stained with Safranin O/Fast green (Sigma-Aldrich, St. Louis, MO, USA) using standard histological technique. Osteoarthritis Research Society International (OARSI) scoring (0-24) was used to assess the severity of cartilage degradation and to confirm congruence between macroscopic (i.e., Outerbridge) and histological (i.e., OARSI) measures of OA severity. The scoring was completed by two independent and blinded observers. The results showed strong consistency. Osteochondral cylinders with OARSI score <12 were considered P-C, and those with OARSI score ≥12 were considered as D-C¹⁸. The information of donors is shown in FIG. 1D.

In DNA-related studies, all samples were sectioned at a thickness of 6 μm. Safranin O/Fast green (Sigma-Aldrich, St. Louis. MO, USA) staining was performed as described above. To assess the severity of cartilage degradation, a semi-quantitative histopathological scoring system recommended by Osteoarthritis Research Society International (OARSI) was performed (on a scale of 0-6). The images were scored by two experienced scorers.

Tissue fixation and embedding in DNA-related studies. In the DNA-studies hereof, human and mouse cartilage samples were first rinsed with PBS, then fixed in 10% buffered formalin (Thermo Fisher Scientific, Waltham, MA) overnight at 4° C., decalcified in formic acid bone decalcifier (StatLab, Mckinney, TX, USA) for 2 days, dehydrated in ethanol (Thermo Fisher Scientific) with ascending concentrations (30-100%), and embedded in paraffin (Thermo Fisher Scientific).

Senescence associated β-Galactosidase staining (SA-β-Gal staining). Cellular senescence was assessed using a senescence-associated β-Galactosidase Staining Kit (BioVision, Milpitas, CA, USA). DAPI staining (Vector Laboratories, Burlingame, CA, USA) was used to counterstain cell nuclei. The staining procedure followed the manufacturer's instructions. The ratio of SA-β-gal positive cells was calculated by dividing blue stained cells (senescent cells) by the total number of cells.

Immunohistochemistry staining (IHC). Cryosectioned samples were prepared as mentioned above. Rehydrated slices were blocked with 10% horse serum (Vector Labs, Burlingame, CA, USA) and then incubated with primary antibodies (Table 1 below) at 4° C. overnight. On the next day, slices were washed with PBS and incubated in biotinylated secondary antibody for an hour, then slices were incubated with horseradish peroxidase (HRP)-conjugated streptavidin for another 0.5 hours and visualized by Vector NovaRED™ peroxidase substrate. Images were acquired with an Olympus CKX41 microscope (Olympus, Shinjuku, Tokyo, Japan), staining intensities were quantified with Image J (National Institutes of Health, Bethesda, MD, USA).

TABLE 1

| Experiment | Antibody | Company | Catlog | Concentration |
|---|---|---|---|---|
| IHC | P16 | Abcam | ab108349 | 1:100 |
| | MMP13 | Abcam | ab39012 | 1:100 |
| | Universal Kit | Vector | PK6200 | / |
| IF | P16 | Abcam | ab54210 | 1:100 |
| | MMP13 | Abcam | ab39012 | 1:100 |
| | COL2 | Abcam | ab34712 | 1:150 |
| | FOXM1 | Abcam | ab180710 | 1:200 |
| | ESR1 | Abcam | ab32063 | 1:200 |
| | OCN | Abcam | ab93876 | 1:200 |
| | DAP1 | Vector | H-1200 | 2-3 drops/slice |
| | Anti-mouse IgG | Abcam | ab150113 | 1:400 |
| | Anti-rabbit IgG | Thermal Fisher | A11037 | 1:400 |
| Western | GAPDH | Cell Signaling | 5174 | 1:2000 |
| Blot | | Technology | | |
| | COL2 | Abcam | ab34712 | 1:2000 |
| | COMP | Abcam | ab74524 | 1:1000 |
| | SOX9 | Abcam | ab185230 | 1:1000 |
| | P16 | Abcam | ab108349 | 1:2000 |
| | P21 | Abcam | ab218311 | 1:1000 |
| | P53 | Abcam | ab26 | 1:1000 |
| | MMP13 | Abcam | ab39012 | 1:1000 |
| | RUNX2 | Cell Signaling | 8486 | 1:1000 |
| | | Technology | | |
| | p-SMAD2/3 | Invitrogen | 44-244G | 1:1000 |
| | ROCK1 | Novos Biologicals | NB100-5659655 | 1:1000 |
| | FOXM1 | Abcam | ab180710 | 1:2000 |
| | ESR1 | Abcam | ab108398 | 1:5000 |
| | P65 (phospho | Abcam | ab76302 | 1:1000 |
| | 5336) | | | |
| | TRPV4 | Abcam | ab191580 | 1:1000 |
| | COL1O | Abcam | ab49945 | 1:1000 |
| | OCN | Abcam | ab93876 | 1:1000 |
| | Mouse IgG | Invitrogen | 31450 | 1:2000 |
| | Rabbit IgG | Healthcare | NA934-1ML | 1:2000 |

For immunohistochemistry (IHC) in the DNA-related studies hereof, rehydrate d slices were processed for antigen retrieval by heating in diluted 1× antigen retrieval solution (eBioscience, San Diego, CA) at 90° C. for 20 minutes. Primary antibodies against target proteins are listed in Table 2 below. The samples were blocked with 10% horse serum (Vector Labs, Burlingame, CA, USA), then incubated with primary antibodies (Supplementary Table S2) at 4° C. overnight. On the next day, slices were washed with PBS and incubated in biotinylated secondary antibody for an hour. Then the slices were incubated with horseradish peroxidase-conjugated streptavidin for another 0.5 hours and visualized by Vector NovaRED™ peroxidase substrate. Hematoxylin was used for counterstaining (Vector Labs). Images were acquired with a Nikon Eclipse E800 upright microscope (Nikon, Melville, NY).

TABLE 2

| | | | | Host | Working |
|---|---|---|---|---|---|
| Experiment | Antibody | Source | Catalog# | Species | Concentration |
| IHC | Estrogen Receptor alpha | Abcam | ab271827 | Rabbit | 1:200 |
| | Universal kit | Vector | PK6101 | NA | NA |
| IF | Estrogen Receptor alpha | Abcam | ab271827 | Rabbit | 1:200 |
| | CDKN2A/p16INK4a | Abcam | ab108349 | Rabbit | 1:200 |
| | Estrogen Receptor alpha | Abcam | ab259427 | Mouse | 1:200 |
| | p21 | Abcam | ab218311 | Rabbit | 1:200 |
| | Goat Anti-Rabbit IgG | Abcam | ab150077 | Goat | 1:500 |
| | H&L (Alexa Floor ®) | | | | |
| | 488) | | | | |
| Western Blot | β-Actin | Cell | 4967S | Rabbit | 1:1000 |
| | | Signaling | | | |
| | | Technology | | | |
| | Estrogen Receptor alpha | Abcam | ab271827 | Rabbit | 1:1000 |
| | CDKN2A/p16INK4a | Abcam | ab108349 | Rabbit | 1:1000 |
| | p21 | Abcam | ab218311 | Rabbit | 1:1000 |
| | Histone H2A.X | Cell | 9718S | Rabbit | 1:1000 |
| | (Phospho Ser139) | Signaling | | | |
| | | Technology | | | |
| | P65 | Abcam | ab32536 | Rabbit | 1:1000 |
| | p-p65 (phospho S536) | Abcam | ab76302 | Rabbit | 1:1000 |
| | Rad50 | Novus | NB100- | Rabbit | 1:500 |
| | | Biologicals | 154SS | | |
| | GREB1 | Novus | NBP3- | Rabbit | 1:1000 |
| | | Biologicals | 10496- | | |
| | | | 100ul | | |
| | Rabbit IgG HRP Linked | GE | NA934- | Donkey | 1:5000 |
| | Whole Ab | healthcare | 1ML | | |

Immunofluorescence staining (IF). Cryosectioned samples were rehydrated with PBS and penetrated by 0.02% triton X-100 (Sigma-Aldrich, St. Louis, MO, USA). After being blocked with 5% BSA, slides were exposed to primary antibodies (Table 1) overnight at 4° C. Secondary antibody incubation was performed for 2 hours, using Alexa Fluor® 488-conjugated (BioRad, Hercules, CA, USA) or Alexa Fluor® 594-conjugated (Life Technologies, Carlsbad, CA, USA), followed by DAPI counterstain. Images were acquired with an Olympus IX2-USB microscope (Shinjuku, Tokyo, Japan).

For immunofluorescence staining (IF) in the DNA-related studies hereof, samples were first penetrated by 0.02% Triton X-100 (Sigma-Aldrich) for 10 minutes. After being blocked with 5% BSA, slides were exposed to primary antibodies (Table 2) overnight at 4° C. Alexa Fluor®488-conjugated Secondary antibody was used (Abcam, Branford, CT, United States). 4′,6-diamidino-2-phenylindole (DAPI)-containing antifade medium (Vector Labs) was utilized to mount the slides. An EVOS M5000 microscope (Thermo Fisher Scientific) was used to image the stained sections.

Isolation of chondrocytes from P-C and D-C. P-C or D-C tissues were minced into 1-2 mm³ morsels with a scalpel blade. Afterward, rinsing medium (Dulbecco's modified Eagle's medium (DMEM, Gibco/Thermo Fisher Scientific, Waltham, MA, USA) with 2% antibiotics-antimycotics (Life Technologies, Carlsbad, CA, USA) was used to wash cartilage morsels for three times. Cartilage morsels were weighed and digested with collagenase type II (1 mg/mL (w/v) in rinsing medium, Worthington Biochemical Corporation, Lakewood, NJ, USA) in a shaker (170 RPM) at 37° C. for 16 hours. The dissociated cells were collected by filtering through a 70 μm mesh, and then cultured in 150 cm² tissue culture flasks with growth medium (GM) (high glucose Dulbecco's modified Eagle's medium, 10% fetal bovine serum (FBS, Life Technologies, Carlsbad, CA, USA), and 1% Antibiotics-Antimycotics). For the first seven days, cells were not disturbed to permit attachment to the flask surface. Following cell adhesion, the medium was changed every three days until cells reached 70-80% confluency. Prior to use, chondrocytes from preserved (P-CHs) and damaged areas (D-CHs) were pooled from six donors (FIG. 1D).

RNA isolation and quantitative real-time PCR. Cells were lysed with QIAzol lysis reagent (Qiagen, Germantown, MD, USA) and total RNA was extracted with the RNeasy Plus Universal Mini Kit (Qiagen). Nanodrop 2000c Spectrophotometer (Thermo Fisher, Waltham, MA, USA) was used to measure total RNA concentration. Reverse transcription was performed by the BioRad iScript cDNA Synthesis Kit (BioRad, Hercules, CA, USA). Quantitative real-time polymerase chain reaction (qRT-PCR) was conducted on CFX384 Touch Real-Time PCR Detection System (BioRad) using SYBR Green Supermix (BioRad). Expression levels of represented chondrogenesis, senescence, fibrosis, degradation, inflammation, hypertrophy and osteogenesis-associated genes were calculated through the ΔΔCt method. Ribosomal protein L13A (RPL13A) was used as the housekeeping gene. Published sequences of forward primers and reverse primer sequences for the genes RPL13A, COL2, SOX9, ACAN, P16, P21, P53, IL6, ILB, COL1, COL3, VCAN, OCN, OPN, OSX, RUNX2, VEGF, ATS4, ATSS, MMP12, MMP13, COL10, ALP and ESR1 were used in the studies hereof.

In the DNA-related studies hereof, to determine the expression level, each gene was normalized to the housekeeping gene (β-Actin). Published sequences for forward primer sequences and reverse primer sequences were used for the genes ESR1, IL6, IL8, IL10, NFKB, and Actin (IDT, Newark, NJ, United States) were used for RT-qPCR studies hereof.

Western blot analysis. Samples were washed three times with PBS and homogenized with cold RIPA buffer (Sigma-Aldrich, St. Louis, MO, USA) with 1× protease inhibitor cocktail (Sigma-Aldrich). Lysates were subsequently centrifuged at 14,000 g for 15 minutes at 4° C. Supernatants were collected and the protein concentration was measured with a BCA kit (Thermo Scientific™ Pierce™ BCA Protein Assay Kit, Waltham, MA, USA). Proteins were fractionated electrophoretically on NuPAGE™ 4-12% Bis-Tris Gel (Invitrogen, Waltham, MA, USA) and then transferred to a polyvinylidene fluoride (PVDF) membrane using the iBlot Dry Blotting System (Invitrogen). Primary antibodies against target proteins are listed in Tables 1 and 2 as described above. Specifically bound primary antibodies were detected using horseradish peroxidase (HRP)-linked secondary antibodies (GE Healthcare Life Sciences, Malborough, MA, USA) and SuperSignal™ West Dura Extended Duration Substrate (ThermoFisher, Waltham, MA, USA). Images were acquired through ChemiDoc™ Touch Imaging System (BioRad). Image J (public domain software for processing and analyzing scientific images) was utilized to semi-quantify the blot images.

Chondrocyte pellet culture. P-CHs or D-CHs were suspended in chondrogenic medium (CM: high glucose DMEM, 50 μg/mL ascorbate 2-phosphate (Sigma-Aldrich, St. Louis, MO, USA), 40 μg/mL L-proline (Sigma-Aldrich), 10 μg/mL ITS+ (Thermo Fisher, Waltham, MA, USA), 10 ng/mL transforming growth factor beta-3 (TGFβ3, Peprotech, Rocky Hill, NJ, USA) and 1% Antibiotics-Antimycotics). Cell suspension was distributed into conical-bottom 96-well plates (ThermoFisher) with 2×10⁵ cells/well and pelleted by centrifuging the plates at 300 g for 10 minutes. CM was changed every two days. Pellets were collected for analyses after 14 days of culture in chondrogenic medium.

Glycosaminoglycan (GAG) assay. Cartilage pellets created by chondrocytes were digested with papain solution (125 μg/mL papain, 100 mM sodium phosphate buffer, 10 mM ethylenediaminetetraacetic acid (EDTA), 10 mM cysteine (Sigma-Aldrich), pH=6.3) and digested in an oven (60° C.) overnight. Digested samples were centrifuged at 12,000 g for 15 minutes, and the supernatant was collected. Afterward, 1,9-dimethylmethylene blue dye-binding assay (Blyscan, Biocolor, United Kingdom) was used to quantitate GAG content in each sample. Picogreen dsDNA assay (Molecular Probes, Tarrytown, NY) was employed for dsDNA quantification.

siRNAs and cell transfection. Two siRNAs respectively targeting human CDKN2A (Assay ID: 118858, Cat: AM51331, ThermoFisher) and ESR1 (Assay ID: 145537, Cat: AM16708, ThermoFisher) were used in this study, with a scrambled siRNA (Cat: AM4611, ThermoFisher) as the negative control. Briefly, chondrocytes at 50-60% confluence were transfected with the siRNA using Lipofectamine RNAiMAX reagent (ThermoFisher) in Opti-MEM medium according to manufacturer's instructions. After 12 hours of incubation, the transfection medium was replaced with basic growth medium (BGM: Phenol red-free DMEM, 1 mM Sodium Pyruvate (Sigma-Aldrich), 1% Antibiotics-Antimycotics). Transfected cells were then used for other studies.

In DNA-related studies hereof, chondrocytes at 70% confluence were transfected with the siRNA using Lipofectamine RNAiMAX reagent (Thermo Fisher Scientific). As described above, after 48 h of incubation, the transfection medium was replaced with medium containing phenol red-free DMEM, 1 mM Sodium Pyruvate (Sigma-Aldrich) and 1% Antibiotic-Antimycotic. Transfected cells were collected after three days.

Overexpression of ESR1 in human chondrocyte. Normal and OA human chondrocytes were transfected with the lentiviral vector carrying ESR1 gene or the control lentivirus carrying mCherry for 12 hours, which were constructed by VectorBuilder (ID: VB900122-1582cgb)(VectorBuilder, Chicago, IL, United States). After transfection, western blot and qPCR were used to verify the stable expression of ESR1 in cells.

RNA sequencing (RNA-Seq) and data processing. RNA-Seq: P-CHs/D-CHs from all six donors were pooled equally. Total RNA was extracted following the method described above. The cDNA library was constructed using TruSeq mRNA kit (Illumina) following the manufacturer's instructions. Briefly, total RNA input was enriched for mRNA and fragmented. Random primers initiate first-strand and second-strand cDNA synthesis. Adenylation of 3′ ends was followed by adapter ligation and library amplification with indexing. Sequencing was performed on a NextSeq500 Illumina sequencing platform, and each group had three replicates.

Data Processing Pipeline: The reverse stranded paired-end RNA-Seq reads, generated by TruSeq mRNA kit, were checked for the presence of adapters and high-quality bases using FastQC (v 0.11.7). These high-quality reads were trimmed for the universal adapter using Cutadapt (v 1.18). The trimmed reads were later mapped against the Ensembl human reference genome (GRCh38 v 97) using the HISAT2 (v 2.1.0) mapping tool. The output file from HISAT2 was converted from SAM format to BAM format using SAMtools (v 1.9). Counts for expressed genes were generated using H T-Seq (v 0.11.2) and were outputted in text format. These count text files were then imported into the Bioconductor R package, edgeR (v 3.24.1). The package was utilized to identify the differentially expressed genes based on the criteria of the genes having an expression count of the absolute value of log base 2 greater than 1 between two experimental conditions and a false discovery rate of less than 0.05. Based on this standard, comparisons of P-CHs vs D-CHs produced 547 differentially expressed genes.

Pathway Analysis: After the differentially expressed genes were identified for each experimental comparison, each list of genes along with their differential expression values were uploaded to Ingenuity Pathway Analysis (IPA). Gene Ontology (GO) and Kyoto Encyclopedia of Genes and Genomes (KEGG) pathway enrichment analysis were performed using Cytoscape (v 3.7) via the plugin ClueGO (v 2.5.3). Upstream molecular prediction was produced by IPA.

Infection of D-CHs Lentiviral activation particles. D-CHs were suspended in GM supplemented with 8 μg/mL polycation polybrene. ERα lentiviral activation particles (Santa Cruz Biotechnology, Cat: sc-400011-LAC) or copGFP control lentiviral particles (Santa Cruz Biotechnology, Cat: sc-108084) were applied at multiplicity of infection (MOI) of 3 particles/cell. In order to enhance the transfection efficiency, lentiviral particles and D-CIs were mixed together and spun at 800 g for 90 minutes in 37° C. After that, D-CHs were resuspended in BGM and cultured in 6-well tissue culture plates for further use.

Encapsulation of P-CHs into gelatin scaffold. The preparation of methacrylated gelatin (GelMA) and photoinitiator was performed according to a previously published procedure. Lin H, Cheng A W, Alexander P G, Beck A M, Tuan R S. Cartilage tissue engineering application of injectable gelatin hydrogel with in situ visible-light-activated gelation capability in both air and aqueous solution. Tissue Eng Part A 2014; 20: 2402-2411, the disclosure of which is incorporated herein by reference. P-CHs were resuspended in 15% (w/v) GeIMA solution at a final density 10×10⁶ cells/mL. The suspension was transferred to a silicone mold, which has a cylindrical void (3.5 mm diameter×2 mm depth). A dental light with wavelength at 395 nm was used to cure the hydrogel. Afterward, P-CH-laden GeIMA scaffolds were maintained in serum-free basic chondrogenic medium (high glucose Dulbecco's modified Eagle's medium, 40 μg/mL L-proline (Sigma), 10 μg/mL ITS+ (Thermo Fisher, Waltham, MA) and 1% Antibiotics-Antimycotics) for the duration of cultures, with medium changes every other day.

Dynamic mechanical loading. Dynamic mechanical loading was conducted using a MechanoActive Transduction and Evaluation (MATE) bioreactor system (Wilsonville, OR, USA) (FIG. 5A). 5% and 20% compressive strains were selected to represent physiological and injurious strain magnitudes, respectively. All constructs were loaded for 1 hour per day (0.2 Hz) for five days. Basic chondrogenic medium was used during the loading.

Animal study. Hind legs from 25- to 30-month-old male C57BL/6 mice were provided by Dr. Ana Maria Cuervo (Albert Einstein College of Medicine) otherwise these tissues would have been treated as waste. Samples were processed, sectioned and stained using similar methods described above. Based on their OARSI score, normal and OA knee joints were classified. Four animals from each group (normal and OA groups) were used for ESR1 IHC and Safranin O/Fast green staining.

Hind legs of ESR1 knockout mice in a C57BL/6 background (Stock No: 026176, KOesr1, 12 weeks) were purchased from Jackson lab (Bar Harbor, Maine, US). Wild-type (WT) mice with matching genders and age were served as the controls.

The protocol was approved by the University of Pittsburgh Institutional Animal Care and Use Committee (IACUC). To create OA models, destabilization of medial meniscus (DMM) surgery was performed on C57BL/6 mice (8-week-old, male, Jackson Laboratory, Bar Harbor, ME) as previously described in Glasson, S. S., Blanchet, T. J., and Morris, E. A. (2007) The surgical destabilization of the medial meniscus (DMM) model of osteoarthritis in the 129/SvEv mouse. Osteoarthritis Cartilage 15, 1061-1069. After surgery, both DMM and sham surgery groups were housed for 8 weeks. The knee joints were fixed and embedded as described above.

Drug treatment. To induce DNA damage in human chondrocytes, doxorubicin (DOX, Sigma-Aldrich) was used. Specifically, when healthy human chondrocytes were at 80% confluency, the culture medium was replaced by DOX-containing medium (Phenol red-free DMEM, 1 mM Sodium Pyruvate, 1% Antibiotic-Antimycotic, and 100 nM DOX). Cells were collected after three days of treatment for different analyses.

Comet assay (single cell gel electrophoresis). Comet assay Kit (Abcam) was utilized to assess the level of DNA damage. The electrophoresis procedure was followed as described in Uehara, M., et al., (2020) Pharmacological inhibition of ataxia-telangiectasia mutated exacerbates acute kidney injury by activating p53 signaling in mice. Sci Rep 10, 4441. DNA damage was evaluated using OpenComet software (v1.3.1). The tail moment (integrated value of tail DNA density multiplied by the migration distance) was used to quantify the DNA damage levels.

Statistical analysis. Graphpad Prism 7 (GraphPad Software, San Diego, CA, USA) was applied for statistical analysis. Intraclass Correlation Coefficient analysis was used to evaluate inter-rater reliability. One-way or two-way analysis of variance (ANOVA) was used for multiple comparisons between groups. P<0.05 was considered as a statically significant difference. Other statistical details for all experiments, including value and definition of n, error bars, and significance thresholds can be found in the description of the Figures.

Statistical analyses in DNA-related studies hereof were performed using GraphPad Prism 9. Data was presented as a box and whiskers plot. The box extended from the 25^{th} to the 75^{th} percentiles, and the whiskers went down to the smallest value and up to the largest value. Unpaired Student's t-test was used for comparison between the two groups. Unless otherwise indicated, data were normalized to the control group (set as 1). P<0.05 was considered statically significant difference, *, p<0.05; **, p<0.01; ***, p<0.001; and ****, p<0.0001.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A system for treating osteoarthritis comprising an injector system and an agent to increase estrogen receptor-α in affected cartilage within a reservoir of the injector system.

2. The system of claim 1 wherein the agent to increase estrogen receptor-α includes at least one of an agent to effect knock-in of an estrogen receptor-α gene, an agent to effect interference with microRNA which suppress estrogen receptor-α gene expression, or an agent to effect enhancement of estrogen receptor-α gene expression.

3. The system of claim 2 wherein the agent to effect knock-in of an estrogen receptor-α gene comprises a plasmid DNA or a viral vector comprising an estrogen receptor-α gene.

4. The system of claim 3 wherein the agent to effect knock-in of an estrogen receptor-α gene further comprises one or more transduction agents.

5. The system of claim 2 wherein the agent to effect interference with microRNA which suppress estrogen receptor-α gene expression comprises siRNA.

6. The system of claim 2 wherein the agent to effect enhancement of estrogen receptor-α gene expression comprises a small molecule compound.

7. The system of claim 6 wherein the agent to effect enhancement of estrogen receptor-α gene expression is a peptide or a selective estrogen receptor modulator.

8. The system of claim 7 wherein the selective estrogen receptor modulator comprises 4-hydroxytamoxifen or 5-aza-2-deoxycytidine.

9. The system of claim 1 wherein the injection system is configured to deliver the agent to increase estrogen receptor-α in affected cartilage locally to the affected cartilage.

10. The system of claim 2 wherein the injection system is configured to deliver the at least one of the agent to effect at least one of knock-in of an estrogen receptor-α gene, the agent to effect interference with microRNA which suppress estrogen receptor-α gene expression, or the agent to effect enhancement of estrogen receptor-α gene expression locally to the affected cartilage.

11. A method of treating osteoarthritis comprising increasing estrogen receptor-α in affected cartilage.

12. The method of claim 11 wherein estrogen receptor-α is increased via delivery of at least one of an agent to effect knock-in of an estrogen receptor-α gene, an agent to effect interference with microRNA which suppress estrogen receptor-α gene expression, or an agent to effect enhancement of estrogen receptor-α gene expression.

13. The method of claim 12 wherein the agent to effect knock-in of an estrogen receptor-α gene comprises a plasmid DNA or a viral vector comprising an estrogen receptor-α gene.

14. The method of claim 13 wherein the agent to effect knock-in of an estrogen receptor-α gene further comprises one or more transduction agents.

15. The method of claim 12 wherein the agent to effect interference with microRNA which suppress estrogen receptor-α gene expression comprises siRNA.

16. The method of claim 12 wherein the agent to effect enhancement of estrogen receptor-α gene expression comprises a small molecule compound.

17. The method of claim 16 wherein the agent to effect enhancement of estrogen receptor-α gene expression is a peptide or a selective estrogen receptor modulator.

18. The method of claim 17 wherein the selective estrogen receptor modulator comprises 4-hydroxytamoxifen or 5-aza-2-deoxycytidine.

19. The method of claim 11 wherein an agent to increase estrogen receptor-α in affected cartilage is delivered locally to the affected cartilage.

20. The method of claim 12 wherein the at least one of the agent to effect at least one of knock-in of an estrogen receptor-α gene, the agent to effect interference with microRNA which suppress estrogen receptor-α gene expression, or the agent to effect enhancement of estrogen receptor-α gene expression is delivered locally to the affected cartilage.
